(19) United States
(12) Patent Application Publication
(10) Pub. No.: US 2012/0294874 A1
(43) Pub. Date: Nov. 22, 2012

(54) Method for Producing T Cell Receptor-Like Monoclonal Antibodies and Uses Thereof

(76) Inventors: Paul Anthony Macary, Singapore (SG); Soh Ha Chan, Singapore (SG); Brendon John Hanson, Singapore (SG)

(21) Appl. No.: 13/510,690

(22) PCT Filed: Nov. 18, 2010
(86) PCT No.: PCT/SG2010/000438

§ 371 Date: May 18, 2012

Related U.S. Application Data
(60) Provisional application No. 61/262,644, filed on Nov. 19, 2009.

(51) Int. Cl. C07K 16/28; C12N 5/20; C12Q 1/70; C12N 15/13; A61P 35/00; C12N 1/21; C12N 5/10; C12N 1/15; C12N 1/19; A61K 39/395; G01N 33/577; C12N 15/85

(52) U.S. Cl. 424/174.1; 435/7.24; 435/339; 435/5; 536/23.53; 435/320.1; 435/252.33; 435/252.35; 435/252.31; 435/252.3; 435/252.34; 435/419; 435/254.11; 435/254.2; 530/389.8; 530/387.9; 530/387.3; 530/391.3; 530/391.7; 424/172.1

(57) ABSTRACT

Methods are provided for producing T cell receptor (TCR) like antibodies that recognize peptides displayed in the context of HLA molecules. Antibodies produced by methods provided herein have the specificity of a TCR and can be used as therapeutic, diagnostic and research reagents. Also provided are TCR-like antibodies which recognize Epstein-Barr virus (EBV) peptides displayed in the context of HLA molecules and are useful for the detection and treatment of EBV and EBV-linked diseases.

### FIELD

The present disclosure relates generally to the field of immunology, and in particular to methods of producing T cell receptor-like antibodies that recognize peptides displayed in the context of HLA molecules.

### BACKGROUND

The mammalian immune system recognizes virus infected cells and tumors through the presentation of small signature peptides derived from viral gene products on the surface of the infected cells in association with HLA-molecules. Class I major histocompatibility complex (MHC) molecules, designated HLA class I in humans, bind and display peptide antigen ligands upon the cell surface. The peptide antigen ligands are derived from either normal endogenous proteins (“self”) or foreign proteins (“nonself”) introduced into the cell. Nonself proteins can be products of malignant transformation or intracellular pathogens such as viruses. In this manner, class I MHC molecules convey information regarding the internal milieu of the cell to immune effector cells, including but not limited to, CD8+ cytotoxic T lymphocytes (CTLs), which are activated upon interaction with “nonself” peptides, thereby lysing or killing the cell presenting such “nonself” peptides.

Class II MHC molecules, designated HLA class II in humans, also bind and display peptide antigen ligands on the cell surface. Unlike class I MHC molecules, which are expressed on virtually all nucleated cells, class II MHC molecules are normally confined to specialized cells, such as B lymphocytes, macrophages, dendritic cells, and other antigen presenting cells which take up foreign antigens from the extracellular fluid via an endocytic pathway. The peptides they bind and present are derived from extracellular foreign antigens, such as bacterial toxins. In this manner, class II molecules convey information regarding the fitness of the extracellular space in the vicinity of the cell displaying the class II molecule to immune effector cells, including but not limited to, CD4+ helper T cells, thereby helping to eliminate such pathogens. The extermination of such pathogens is accomplished by both helping B cells make antibodies against microbes, as well as toxins produced by such microbes, and by activating macrophages to destroy ingested microbes.

Class I MHC molecules alert the immune response to disorders within host cells. Peptides which are derived from viral- and tumor-specific proteins within the cell are loaded into the class I molecule's antigen binding groove in the endoplasmic reticulum of the cell and subsequently carried to the cell surface. Once the class I MHC molecule and its loaded peptide ligand are on the cell surface, the class I molecule and its peptide ligand are accessible to cytotoxic T lymphocytes (CTL). CTLs survey the peptides presented by the class I molecule and destroy those cells harboring ligands derived from infectious or neoplastic agents within that cell.

The majority of proteins produced by a cell reside within intracellular compartments, thus preventing their direct recognition by antibody molecules. The abundance of intracellular proteins that are available for degradation by proteasome-dependent and independent mechanisms yields an enormous source of peptides for surface presentation in the context of the MHC class I system (Rock et al., (2004)). A new class of antibodies that specifically recognizes HLA-restricted peptide targets (epitopes) on the surface of virus-infected or tumor cells would significantly expand the therapeutic repertoire.

The most widely used method for visualizing or targeting virus infected cells or tumors involves the production of antigen-specific T-lymphocyte cell lines in vitro that express T cell receptors specific for the virus peptide/HLA complex. However, the generation of these antigen-specific human cell lines requires a high degree of technical proficiency, and such cell lines are extremely unstable in vitro. An alternative to developing antigen-specific T-lymphocyte cell lines is to make TCR-like monoclonal antibodies that have similar or overlapping specificities as T-lymphocytes. This requires antibodies that have fine specificities for small antigenic peptides presented on the surface of human cells in association with HLA-molecules. This too represents a significant technical challenge because, as a whole, HLA molecules of human origin are extremely antigenic when used as an immunogen in mice. Thus, TCR-like mouse monoclonals potentially represent a small fraction of the possible antibody repertoires induced in mice as a result of immunization of human HLA complexes plus antigenic peptide of interest.

TCR-like monoclonal antibodies have been produced using a standard hybridoma approach or by employing phage antibody libraries. However, this type of antibody remains extremely rare and their affinities when made by phage display are predictably low. The use of antibodies recognizing. MHC-peptide complexes has previously been described (see e.g., Reiter, U.S. Patent App. Pub. No. 2004/0191260 A1, filed Mar. 26, 2003; Andersen et al., U.S. Patent App. Pub. No. 2002/0150914 A1, filed Sep. 19, 2001; Hoogenboom et al., U.S. Patent App. Pub. No. 2003/0223994, filed Feb. 20, 2003; and Reiter et al., PCT App. Pub. No. WO 03/068201, filed Feb. 11, 2003). However, the disclosed processes employ phage display libraries that do not produce a whole, ready-to-use antibody product. In addition, the majority of these antibodies were isolated from bacteriophage libraries as Fab fragments (Cohen et al., J Mol Recognit, 16:324-332 (2003); Held et al., (2004); Chames et al., J Immunol, 169:1110-1118 (2002)), which have not been examined for anti-viral or anti-tumor activity since they do not activate innate immune mechanisms (e.g., complement-dependent cytotoxicity (CDC)) or antibody-dependent cellular cytotoxicity (ADCC).

To date, there has been no demonstrated production of antibodies capable of staining tumor or virally infected cells in a robust manner, implying that previously produced antibodies are of low affinity and/or specificity. The immunogens employed in prior methods have included MHC, which had been “enriched” for a particular peptide. The immunogen thus contained a pool of MHC-peptide complexes and was not loaded solely with the peptide of interest. In addition, there has been no conceited effort in these methods to maintain the structure of the three dimensional epitope formed by the HLA-peptide complex, which is essential for generation of the appropriate antibody response.

Thus, antibodies with true MHC-restricted specificity of T cells are very rare. Such antibodies have proven difficult to generate by conventional hybridoma techniques because B cells are not educated to be self-MHC-restricted (Porgador et al., Immunity, 6:715-726 (1997); Dadaglio et al., Immunity, 6:727-738 (1997); Aharoni et al., Nature, 351:147-150 (1991); Krogsgaard et al., J Exp Med, 191:1395-1412 (2000)). Previously described TCR-like antibodies generally have had relatively low binding affinities for their corresponding MHC-peptide complexes, which is indicative of reduced antibody selectivity. In fact, the limited antibody binding affinities have thus far made it impractical to isolate B cells of interest by conventional methods (U.S. Patent App. Pub. No. 2009/0226474). Thus, there is a need in the art for methods of producing TCR-like monoclonal antibodies that are HLA-restricted and have high binding affinities.

The Epstein-Barr virus (EBV) is a ubiquitous human herpes virus which is found as a predominantly asymptomatic infection in all human communities. EBV has been linked to numerous human tumors of diverse tissue origin. EBV-positive tumors are characterized by active expression of viral gene products, consistently observed in NPC biopsies and other EBV malignancies which include the elements of EBNA-1, LMP-1, and LMP-2A.

Although EBV has been well studied and researched, there is still no antibody in the art with specificity for at least one EBV derived peptide expressed in association with human HLA on the surface of EBV infected cells.

In particular, while existing tetramer technology can be utilized to study EBV-specific T-lymphocytes generated as a result of natural infection, there are no current methodologies that allow the visualization, quantification and analysis of the TCR ligands expressed on the surface of EBV infected cells and tumors. These limitations severely impede the ability to directly address the impact of candidate immune evasion mechanisms on the processing and presentation of EBV antigens in infected cells and APC. These limitations also restrict targeting of tumor cells in situ based on their association with EBV.

Thus, there is also a need in the art for antibodies with binding specificity for EBV and/or EBV derived peptides.

### SUMMARY

The present disclosure addresses long-felt needs in the field of immunology by providing novel, efficient and effective methods for producing high affinity T cell receptor-(TCR) like antibodies. Moreover, the present disclosure provides TCR-like antibodies that are specific to EBV and/or EBV infected cells.

According to one aspect, the present disclosure provides methods for producing a T cell receptor-like antibody, the method comprising the steps of: forming an immunogen comprising a monomeric MHC-peptide complex; administering an effective amount of the immunogen to a host for eliciting an immune response to the peptide within the MHC-peptide complex; selecting a B cell specific to the peptide in the MHC-peptide complex; forming a hybridoma by fusing the B cell with an immortalized cell; and isolating an antibody produced by the hybridoma.

According to further aspects, the present disclosure provides a TCR-like antibody produced according to the method comprising the steps of: forming an immunogen comprising a monomeric MHC-peptide complex; administering an effective amount of the immunogen to a host for eliciting an immune response to the peptide within the MHC-peptide complex; selecting a B cell specific to the peptide in the MHC-peptide complex; forming a hybridoma by fusing the B cell with an immortalized cell; and isolating an antibody produced by the hybridoma.

According to further aspects, the present disclosure provides methods for producing a hybridoma, the method comprising the steps of: forming an immunogen comprising a monomeric MHC-peptide complex; administering an effective amount of the immunogen to a host for eliciting an immune response to the peptide within the MHC-peptide complex; selecting a B cell specific to the peptide in the MHC-peptide complex; forming a hybridoma by fusing the B cell with an immortalized cell; and isolating the hybridoma.

According to another aspect, the present disclosure provides an isolated hybridoma cell line having ATCC Accession Number PTA-10351.

According to further aspects, the present disclosure provides methods for detecting the presence of at least one EBV infected cell in a subject, the method comprising: contacting a TCR-like antibody or fragment thereof with a sample obtained from a subject; and detecting binding of the antibody to the EBV infected cell.

According to further aspects, the present disclosure provides an isolated nucleic acid molecule encoding: at least one heavy chain of the antibody or a fragment thereof according to claim 5, wherein the heavy chain comprises an amino acid sequence selected from the group consisting of SEQ ID NOS:4 to 6, a variant, mutant or fragment thereof; and/or at least one light chain of a TCR-like antibody or fragment thereof, wherein the light chain comprises an amino acid sequence selected from the group consisting of SEQ ID NOS:1 to 3, a variant, mutant or fragment thereof.

According to further aspects, the present disclosure provides a method of detecting the presence of EBV, a method of treating at least one EBV-linked disease, and the antibody or fragment thereof of the present disclosure for use as medicament and in a kit.

### BRIEF DESCRIPTION OF THE FIGURES

FIG. 1 shows a schematic illustration of the experimental procedure used for the production of at least one TCR-like monoclonal antibody. Human Major Histocompatibility Complex (MHC) class-I heavy chains (HC) and light chains (LC) are expressed as recombinant proteins and refolded into HLA monomers in the presence of an antigenic peptide YLLEMLWRL (SEQ ID NO:7) from LMP-1 of EBV. The monomers are then purified by fast protein liquid chromatography (FPLC) and used as immunogens in Balb/C mice. Splenic B lymphocytes with desired antigenic specificities are purified using immuno-magnetic beads prior to polyethylene glycol (PEG) mediated fusion with the myeloma cell line NS1. The resulting hybridoma clones are screened by flow cytometry using peptide pulsed HLA-A201 expressing human cell lines.

FIG. 2 shows the expression and purification of HLA heavy and light chains to form a monomer. FIG. 2(A) shows the result of an SDS-PAGE where the protein content of heavy chain (HC) and light chain (LC) inclusion bodies were analyzed. Recombinant HLA-A201 heavy and light chains were expressed in BL21 E. coli. The heavy and light chains were isolated as inclusion bodies and dissolved in 8M urea. FIG. 2(B) shows the results of the (i) FPLC profile and (ii) SDS-PAGE analysis of the purified folded monomers. Heavy chain, light chain and the LMP-1 peptide (YLLEMLWRL; SEQ ID NO:7) were refolded into complete HLA-A2/LMP-1 epitope complexes in vitro. Anion-exchange chromatography was used to purify the monomers. Peak A at 19 minutes contained the light chain (β2m) only, whereas peak B at 36-37 minutes contained both the heavy chain (35 kD) and light chain (12kD). FIG. 2(C) shows the results of a non-denaturing native gel where, fractions 1 and 2, collected from peak B, were pooled and analyzed. The gel was immunoblotted with the anti-HLA conformation specific monoclonal antibody w6.32. These results confirmed that the purified monomer was correctly folded. FIG. 2(D) shows the analysis of HLA-A2/LMP-1 epitope complexes that were tetramerized and tested for their capacity to bind LMP-1 specific CD8+ T cells. The tetramer binding to CD8+ T cells from an EBV positive donor (left panel) is shown versus a control tetramer (right panel). These results confirm that the HLA-A2/LMP-1 epitope complexes had folded correctly prior to their use as immunogens.

FIG. 3 shows a schematic illustration of the procedure involved in the pre-selection of B cells from immunized mice using immunomagenetic selection. In summary, biotynylated HLA monomers/immunogens bind to specific B cell receptors found on the surface of B cell specific to the monomer. Anti-biotin coated Miltenyi magnetic-activated cell sorting (MACs) beads (i.e., Miltenyi-Biotec, Singapore) were used to isolate bound B cells on magnetic columns prior to myeloma fusion.

FIG. 4 shows the result of screening B cell hybridomas for TCR-like monoclonal antibodies with HLA-A201/LMP-1 specificity. The binding of antibodies in the supernatant of the hybridoma clone, LMP1#226 to C1R.A2 cells pulsed with LMP-1 peptide (grey histogram) was compared by flow cytometry with C1R.A2 cells pulsed with the HLA-A201 restricted Influenza A peptide GILGFVFTL (SEQ ID NO:8; black histogram). The grey histogram shows the staining pattern of hybridoma clone LMP1#226.

FIG. 5 shows a pie chart illustrating that the pre-selection of B cells for desired specificity significantly enhances the percentage of HLA-A201/LMP-1 specific hybridomas versus unselected B cells. Unselected B cells are compared with B cells selected on the basis of their binding to HLA-A201/LMP-1 monomers prior to fusion with myeloma cells. Unselected B cells generated very low numbers of HLA-A201/LMP-1 specific hybridomas compared to B cells selected for their binding capacity to the HLA-A201/LMP-1 monomers. The selected cells were also found to have stable phenotypes with better overall specificities compared to positive clones from the unselected hybridoma pool.

FIG. 6 shows the result of the immunoglobulin isotype test for anti HLA-A201/LMP-1 monoclonal antibodies. The isotype profile for hybridoma clone LMP1#226 shown is identical to that seen for 24 other hybridoma clones with similar specificities. HC=heavy chains, LC=light chains.

FIG. 7 shows the experimental dose response curve for TCR-like monoclonal antibodies from (i) LMP1#226 (anti HLA-A201/LMP-1) and (ii) BB7.2 (anti HLA-A2) at increasing concentrations of antigen. Antigen concentrations ranged from 7.5-100 ng/ml. The data points were fitted using four-parameter logistics. The summed square deviations were no higher than 0.393 for all data points expressed.

FIG. 8 shows that anti-HLA-A201/LMP-1 monoclonal antibodies can be used to detect EBV infected cells. In particular, 2E, RAJI, (both HLA-A2 negative B cell lines), CF986 (HLA-A201 B cell line) and CM960 (HLA-A203 B cell line) were super-infected with EBV for 24 hours. The cell lines were then labeled with a polyclonal anti-HLA class-I and antibody from clone LMP1#226 and analyzed by confocal microscopy. EBV infection of the HLA-A201 B cell line (i.e., CF986) led to binding of antibody from clone LMP1#226 and the resulting staining pattern co-localized with the polyclonal anti-HLA. No significant binding was detected in the RAJI or HLA-A203 positive B cell lines treated under similar conditions.

FIG. 9 shows that anti HLA-A201/LMP-1 recognizes a LMP-1 epitope presented on HLA-A201, HLA-A206 and HLA-A207. Flow cytometry was used to analyze binding to six B cell lines pulsed with LMP-1 peptide. Cell lines, CF801 and CM803 (HLA-A201) showed significant binding along with cell lines, CM304 and CF1007 (HLA-A206 and HLA-A207 respectively). Cell lines, CM392 and CM960 (HLA-A203) did not exhibit any binding. This result suggests that these HLA-A2 polymorphisms result in varied epitope specificities.

FIG. 10(A) shows the detection of LMP-1 in latent EBV infected human B cells. 1×10⁶ EBV transformed B cells were screened for the presence of LMP-1 using a commercially available anti-LMP-1 antibody (Acc Chem & Sci Co, USA) (top panel). LMP-1 was detected in 50% of human EBV B cell lines. A monoclonal antibody specific for human HLA (HLA-HC) was used as a loading control. FIG. 10(B) shows the results of flow cytometry based detection of surface HLA-A201/LMP-1 complexes on latent EBV infected B cell lines. EBV immortalized human B cells were stained with 0.5 μg of anti-HLA-A201/LMP-1 at 40° C. for 20 minutes. Cells were washed twice in ice cold phosphate buffered saline prior to incubation with 0.5 μg of goat-anti-mouse IgG-Alexa-Fluor 488 (Jackson Labs, USA) at 4° C. for 20 minutes. Stained cells were washed twice in ice cold phosphate buffered saline, fixed with 500 μl of 1% paraformaldehyde and analyzed on a Becton-Dickinson FACs Calibur Flow Cytometer. The results indicate that LMP-1 expression was detected in latently infected human B cells.

FIG. 11 shows that anti-HLA-A201/LMP-1 monoclonal antibodies block cytotoxic killing mediated by T cells expressing T cell receptors specific for same peptide/HLA complexes (HLA-A201/LMP-1). FIG. 11(A) shows the tetramer staining results of an LMP-1 peptide specific CD8+ T cell line (generated in house), in which more than 30% of cells were expressing a T cell receptor specific for HLA-A201/LMP-1. FIG. 11(B) shows the results of a Cr⁵¹ release assay employed to test for an overlap in the specificity of LMP-1 peptide specific CD8+ T cell line with antibodies from clone LMP1#226. Pre-incubation with peptide pulsed targets with antibodies from clone LMP1#226 induced a significant reduction in the killing activity of the CD8+ T cell line. These results suggest that the cells and the antibody share overlap in their binding specificities to HLA-A201/LMP-1 complexes.

FIG. 12 shows that anti HLA-A201/LMP-1 monoclonal antibodies induce antibody-dependent cell-mediated cytotoxicity (ADCC) activity against CIR.A2 targets pulsed with LMP-1 peptide versus controls. Splenic NK cell effectors were used with Cr⁵¹ labeled CIR.A2 cells as targets for this assay. The LMP-1 derived peptide was titrated onto the CIR.A2 target cells with unpulsed cells used as the negative control. Results are expressed as percent specific lysis of LMP-1 peptide pulsed targets versus controls (mean of triplicate wells +/−SEM).

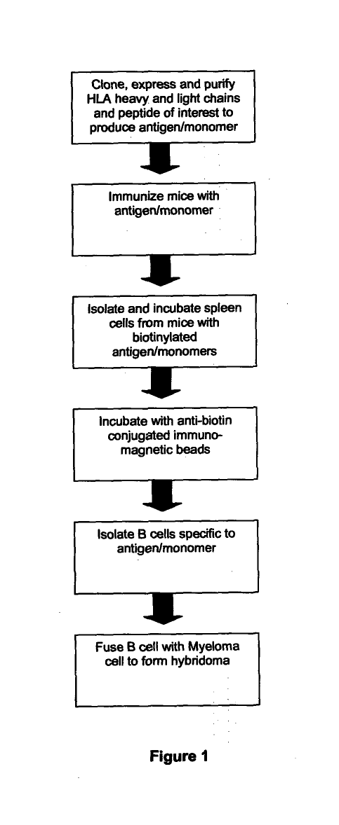

### DETAILED DESCRIPTION

The present disclosure relates generally to antibodies having T cell receptor specificity and far higher affinity than known antibodies with similar specificity. The disclosure also relates generally to hybridomas capable of producing such antibodies and T cell receptor-like antibody conjugates with identifiable and/or therapeutic moieties, such as immunotoxins and immunolabels. Further aspects of the present disclosure provide for methods of making such antibodies, hybridomas, and conjugates, as well as polynucleotides encoding such antibodies and conjugates, and methods of using such conjugates in the detection and treatment of EBV-linked disorders.

The descriptions of various aspects of the present invention are presented for purposes of illustration, and are not intended to be exhaustive or to limit the claimed methods to the forms disclosed. Persons skilled in the relevant art can appreciate that many modifications and variations are possible in light of the aspect teachings.

It should be noted that the language used herein has been principally selected for readability and instructional purposes, and it can not have been selected to delineate or circumscribe the inventive subject matter. Accordingly, the disclosure is intended to be illustrative, but not limiting, of the scope of claimed methods.

As used in this specification and the appended claims, the singular forms “a”, “an” and “the” include plural referents unless the content clearly dictates otherwise. Thus, for example, reference to “an antibody” includes a combination of two or more antibodies, and the like.

Any terms not directly defined herein shall be understood to have the meanings commonly associated with them as understood within the art of the present disclosure. Certain terms are discussed herein to provide additional guidance to the practitioner in describing the compositions, devices, methods and the like of aspects of the present disclosure, and how to make or use them. It will be appreciated that the same thing can be said in more than one way. Consequently, alternative language and synonyms can be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the present disclosure herein.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “treating” refers to any indicia of success in the treatment or amelioration or prevention of the disease, condition, or disorder, including any objective or subjective parameter such as abatement, remission, diminishing of symptoms or making the disease condition more tolerable to the patient, slowing in the rate of degeneration or decline, or making the final point of degeneration less debilitating. The treatment or amelioration of symptoms can be based on objective or subjective parameters, including the results of an examination by a physician. Accordingly, the term “treating” includes the administration of the compounds or agents of the invention to prevent or delay, to alleviate, or to arrest or inhibit development of the symptoms or conditions associated with a disease, condition or disorder as described herein. The term “therapeutic effect” refers to the reduction, elimination, or prevention of the disease, symptoms of the disease, or side effects of the disease in the subject. “Treating” or “treatment” using the methods of the invention includes preventing the onset of symptoms in a subject that can be at increased risk of a disease or disorder associated with a disease, condition or disorder as described herein, but does not yet experience or exhibit symptoms, inhibiting the symptoms of a disease or disorder (slowing or arresting its development), providing relief from the symptoms or side effects of a disease (including palliative treatment), and relieving the symptoms of a disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

As used herein, the terms “patient”, “subject” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

As used herein, the term “therapeutically effective amount” or “an amount effective to reduce or eliminate viral infection” refers to an amount of TCR-like antibody that is sufficient to prevent viral infection or to alleviate (e.g., mitigate, decrease, reduce) at least one of the symptoms associated with viral infection. It is not necessary that the administration of the compound eliminate the symptoms of viral infection, as long as the benefits of administration of compound outweigh the detriments.

As used herein, the term “pharmaceutically acceptable carrier” means a carrier that is useful in preparing a pharmaceutical composition that is generally safe, non-toxic, and desirable, and includes carriers that are acceptable for veterinary use as well as for human pharmaceutical use. Such carrier can be solid, liquid, semisolid, or, in the case of an aerosol composition, gaseous.

As used herein, the term “sufficient amount” means an amount sufficient to produce a desired effect, e.g., an amount of TCR-like antibody sufficient to reduce the incidence of EBV infection or EBV induced tumorigenesis in a subject.

As used herein, the term “isolated” means a biological component, such as a nucleic acid, peptide or protein, that has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

As used herein, the term “sample,” is used in its broadest sense. A biological sample suspected of containing nucleic acids encoding at least one EBV derived peptide, or fragments thereof, or at least one EBV derived peptide itself can comprise a bodily fluid, an extract from a cell, chromosome, organelle, or membrane isolated from a cell, a cell, genomic DNA, RNA, or cDNA (in solution or bound to a solid support), a tissue, a tissue print, and the like.

As used herein, the term “TCR-like monoclonal antibody” refers to an antibody that behaves similarly to a T cell receptor (TCR) antibody. In particular, the term “TCR-like monoclonal antibody” refers to an antibody that selectively recognizes MHC-bound peptides.

A person skilled in the art will appreciate that the present disclosure can be practiced without undue experimentation according to the methods given herein. The methods, techniques and chemicals are as described in the references given or from protocols in standard biotechnology and molecular biology textbooks.

### Major Histocompatibility Complexes (MHCs):

As used herein, the term “MHC” will be understood to refer to the major histocompability complex, which is defined as a set of gene loci specifying major histocompatibility antigens. As used herein, the term “HLA” will be understood to refer to human leukocyte antigens, which is defined as the histocompatibility antigens found in humans. As used herein, “HLA” is the human form of “MHC”.

The major histocompatibility complex (MHC) is a complex of antigens encoded by a group of linked loci, which are collectively termed H-2 in the mouse and HLA in humans. The two principal classes of the MHC antigens, class I and class II, each comprise a set of cell surface glycoproteins which play a role in determining tissue type and transplant compatibility. In transplantation reactions, cytotoxic T cells (CTLs) respond mainly against foreign class I glycoproteins, while helper T cells respond mainly against foreign class II glycoproteins.

MHC class I molecules are expressed on the surface of nearly all cells. These molecules function in presenting peptides which are mainly derived from endogenously synthesized proteins to CD8+ T cells via an interaction with T cell receptors. The class I MHC molecule is a heterodimer composed of a 46-kDa heavy chain which is non-covalently associated with the 12-kDa light chain β2 microglobulin. In humans, there are several MHC haplotypes, such as, for example, HLA-A, HLA-B, HLA-C, HLA-DPA1, HLA-DPB1, HLA-DQA1, HLA-DQB1, HLA-DRA, and HLA-DRB1.

Recombinant MHC class I and class II complexes which are soluble and which can be produced in large quantities are described in, for example, Denkberg, et al., Eur J Immunol, 30:3522-3523 (2000); Denkberg, et al., J Immunol, 167:270-6 (2001); Garboczi, et al., Proc Natl Acad Sci USA, 89:3429 (1992); Uger, et al., J Immunol, 160:1598 (1998) and further in U.S. patent application Ser. No. 09/534,966 and PCT App. No. PCT/IL01/00260 (published as PCT App. Pub. No. WO 01/72768), all of which are incorporated herein by reference. Soluble MHC class I molecules are available or can be produced for any of the MHC haplotypes, such as, for example, HLA-A, HLA-B, HLA-C, HLA-DPA1, HLA-DPB1, HLA-DQA1, HLA-DQB1, HLA-DRA, and HLA-DRB1, following, for example the teachings of PCT/IL01/00260, as their sequences are known and can be found at the kabat data base, at immuno.bme.nwu.edu/, the contents of the site is incorporated herein by reference.

### MHC-Binding Peptides

Class I, MHC-restricted peptides (also referred to herein interchangeably as HLA-restricted antigens, HLA-restricted peptides, MHC-restricted antigens) are typically 8 to 10-amino acids long and bind to MHC's heavy chain groove via two or three anchor residues that interact with corresponding binding pockets in the MHC molecule. The β2 microglobulin chain plays an important role in MHC class I intracellular transport, peptide binding, and conformational stability. For most class I molecules, the formation of a heterodimer consisting of the MHC class I heavy chain, peptide (self or antigenic) and β2 microglobulin is required for biosynthetic maturation and cell-surface expression.

As used herein, the term “peptide” refers to native peptides (either degradation products or synthetically synthesized peptides) and further to peptidomimetics, such as peptoids and semipeptoids which are peptide analogs, which can have, for example, modifications rendering the peptides more stable while in a body, or more immunogenic. Such modifications include, but are not limited to, cyclization, N terminus modification, C terminus modification, peptide bond modification, backbone modification, and residue modification. Methods for preparing peptidomimetic compounds are well known in the art and are specified in Quantitative Drug Design, C. A. Ramsden Gd., Chapter 17.2, F. Choplin Pergamon Press (1992), which is incorporated by reference as if fully set forth herein.

As used herein, the term “amino acid” is understood to include the 20 naturally occurring amino acids; those amino acids often modified post-translationally in vivo, including for example hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids. Further elaboration of the possible amino acids usable according to the invention are provided throughout the present disclosure.

As used herein, the term “conservative amino acid substitution” means an amino acid substitution in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in a protein is preferably replaced with another amino acid residue from the same side chain family. Alternatively, in another aspect, mutations can be introduced randomly along all or part of a coding sequence, such as by saturation mutagenesis, and the resultant mutants can be screened for biological activity to identify mutants that retain activity. Following mutagenesis of the protein sequences, the encoded protein can be expressed recombinantly and the activity of the protein can be determined.

The peptides of interest can be associated with an infectious or diseased state, a tumorigenic state, or the peptide of interest can be specific to a particular organ or tissue. The presentation of the peptide in context of an MHC molecule can be novel to virally infected cells or tumor cells.

As used herein, the term “derivative,” refers to the chemical modification of at least one EBV derived peptide, or a polynucleotide sequence encoding at least one EBV derived peptide, or of a polynucleotide sequence complementary to a polynucleotide sequence encoding at least one EBV derived peptide. Chemical modifications of a polynucleotide sequence can include, for example, replacement of hydrogen by an alkyl, acyl, or amino group. A derivative polynucleotide encodes a polypeptide which retains at least one biological or immunological function of the natural molecule. A derivative polypeptide is one modified by glycosylation, pegylation, or any similar process that retains at least one biological or immunological function of the polypeptide from which it was derived.

A “variant” of at least one EBV derived peptide, as used herein, refers to an amino acid sequence that is altered by one or more amino acids. The variant can have “conservative” changes, wherein a substituted amino acid has similar structural or chemical properties (e.g., replacement of leucine with isoleucine). More rarely, a variant can have “nonconservative” changes (e.g., replacement of glycine with tryptophan). Analogous minor variations can also include amino acid deletions or insertions, or both. Guidance in determining which amino acid residues can be substituted, inserted, or deleted without abolishing biological or immunological activity can be found using computer programs well known in the art, for example, DNASTAR software.

As used herein, the phrase “EBV derived peptide” refers to any peptide that can be derived from EBV. In particular, the EBV derived peptides can include but are not limited to Epstein-Barr nuclear antigen (EBNA)-1, EBNA-2, EBNA-3A, EBNA-3B, EBNA-3C, EBNA-leader protein (EBNA-LP), latent membrane proteins LMP-1, LMP-2A and LMP-2B, EBV-EA, EBV-MA, EBV-VCA and the like.

The term “fragment” as used herein, refers to an incomplete or isolated portion of the full sequence of at least one EBV derived peptide which comprises the active site(s) that confers the sequence with the characteristics and function of the peptide. In particular, it can be shorter by at least one nucleotide or amino acid and can be an immunogenic fragment. For example a fragment of LMP-1 comprises the active site(s) that enable the LMP-1 antibody or fragment thereof to recognize LMP-1.

### Methods of Producing MHC-Peptide Complexes

In humans, the MHC restriction of at least one epitope can be dependent on at least one particular human leukocyte antigen (HLA) expressed by at least one cell presenting the antigen. Different HLA types include HLA-A, HLA-B, HLA-C, HLA-DPA1, HLA-DPB1, HLA-DQA1, HLA-DQB1, HLA-DRA, and HLA-DRB1.

In particular, the antibodies of the present disclosure are capable of recognizing epitopes that are HLA-A2 restricted. Approximately 50% of the general population expresses the MHC class I molecule HLA-A2, an HLA-A serotype. In particular, the subtype can identify gene products of many HLA-A*02 alleles, comprising HLA-A*0201, *0202, *0203, *0206, and *0207 gene products. There can be distinct differences in the subtypes between Caucasian and Asian populations. Whereas more than 95% of the HLA-A2 positive Caucasian population is HLA-A0201 the HLA-A2 positive, the Chinese population can be broken down into 23% HLA-A0201; 45% HLA-A0207; 8% HLA-A0206; 23% HLA-A0203.

As used herein, the term “monomer” means an antigen, a heavy chain and/or light MHC chain. For example, in the present disclosure, the monomer used in each of the EXAMPLES below comprises LMP-1 and at least one HLA heavy and light chain. In particular, the monomer can comprise SEQ ID NO:7 or a fragment thereof, a heavy and a light chain. The term “monomer” can be used interchangeably in the present disclosure with the term “HLA-A2/LMP-1 epitope complex”, “HLA-LMP-1 peptide monomers”, “HLA-peptide monomers,” or “HLA monomers”.

As used herein, the term “multimer” will be understood to include two or more copies of the MHC-peptide complex which are covalently or non-covalently attached together, either directly or indirectly. The MHC molecules of the complexes can be produced by any methods known in the art. Examples of MHC production include but are not limited to endogenous production and purification, or recombinant production and expression in host cells. In one aspect, the MHC heavy chain and β2m molecules are expressed in E. coli and folded together with a synthesized peptide. In another aspect, the MHC-peptide complex can be produced as the genetically-engineered single-chain trimer (or the single-chain dimer plus MHC heavy chain) described above.

Immunizing with multimeric MHC-peptide complexes has several disadvantages relative to immunizing with monomeric MHC-peptide complexes. First, the process of forming multimers is laborious, time-consuming, and expensive. The primary disadvantage, however, is a reduction in antibody selectivity due to the introduction of numerous additional antigens (e.g., streptavidin, etc.). Any advantage resulting from multimeric MHC-peptide complex stability is outweighed by these deleterious effects.

The immune system is controlled and regulated by the T cell receptor (TCR), which specifically recognizes peptide-MHC complexes. In recent years, many viral- and cancer-associated MHC-restricted peptides have been isolated and because of their highly restricted fine specificity, they are desirable targets for novel approaches in immunotherapy and immunodiagnosis. Antibodies that are able to recognize viral- or cancer-associated MHC-peptide complexes, with the same specificity as the T cell antigen receptor, would be valuable reagents for studying antigen presentation by virally infected cells and tumor cells, for visualizing MHC-peptide complexes on such cells, and eventually for developing new targeting agents for viral infections and cancer immunotherapy and immunodiagnosis.

Only a few publications have reported the generation of self-MHC-restricted antibodies by conventional means such as the hybridoma technology (Porgador et al., Immunity, 6:715-726 (1997); Dadaglio et al., Immunity, 6:727-738 (1997); Aharoni et al., Nature, 351:147-150 (1991); Krogsgaard et al., J Exp Med, 191:1395-1412 (2000)). The major reason for these past difficulties can be found in the molecular nature and the resolved structures of MHC-peptide complexes. More specifically, the peptides are deeply buried inside the MEC-binding groove and therefore they are presented as extended mosaics of peptide residues intermingled with the MHC residues.

The present disclosure further relates to an immunogen useful in the production of a T cell receptor-like antibody. The immunogen comprises a monomer of a MHC-peptide complex, wherein the MHC-peptide complex is capable of retaining its 3-dimensional form for a period of time sufficient to elicit an immune response in a host such that antibodies that recognize a three-dimensional presentation of the peptide in the binding groove of the MHC molecule are produced. The antibodies so produced are capable of differentiating the MHC-peptide complex from the MHC molecule alone, the peptide alone, and a complex of MHC and irrelevant peptide. The peptide of the specific MHC-peptide complex can be associated with an infections state, tumorigenic state, and/or a disease state, or the peptide of the specific MHC-peptide complex can be specific to a particular organ or tissue. Alternatively, the presentation of the peptide of the specific MHC-peptide complex in the context of an MHC molecule can be novel to EBV infected cells or EBV associated tumor cells. The peptide of the specific MHC-peptide complex can comprise an EBV derived peptide, a variant, mutant or fragment thereof. In particular, the EBV derived peptide can be LMP-1, a variant, mutant or fragment thereof. More particularly, the LMP-1 comprises the amino acid sequence of SEQ ID NO:7, a variant, mutant or fragment thereof.

The present disclosure also relates to methods for producing TCR-like antibodies that recognize peptides displayed in the context of HLA molecules, wherein the peptide is associated with an infection or disease state or is tumorigenic. These antibodies have the specificity of a T cell receptor (TCR) such that the molecules can be used as therapeutic, diagnostic and research reagents. In certain aspects, the TCR-like antibodies of the present disclosure have a higher binding affinity than a T cell receptor.

Also described herein are methods for the production of an immunogen. The immunogen comprises a MHC-peptide complex, wherein the 3-dimensional presentation of the peptide in the binding groove is the epitope recognized with high specificity by the antibody. The immunogen can be any form of a stable MHC-peptide complex that can be utilized for immunization of a host capable of producing antibodies to the immunogen, and the immunogen can be produced by any methods known to those skilled in the art. The immunogen is used in the construction of an agent that will activate a clinically relevant cellular immune response against the tumor cell which displays the particular MHC-peptide complex. The immunogen can be produced in a manner so that it is stable, or it can be modified by various means to make it more stable.

In one aspect, the immunogen is produced by a cell-based approach through genetic engineering and recombinant expression, thus significantly increasing the half-life of the complex. The genetically engineered and recombinantly expressed MHC-peptide complex can be chemically cross-linked to aid in stabilization of the complex. Alternatively, or in addition to chemical cross-linking, the MHC-peptide complex can be genetically engineered such that the complex is produced in the form of a single-chain trimer. In this method, the MHC heavy chain, β2 microglobulin and peptide are all produced as a single-chain trimer that is linked together. Methods of producing single-chain trimers are known in the art and are disclosed particularly in Yu et al., Immunol, 168:3145-3149 (2002). Other methods involve forming a single-chain dimer in which the peptide-β2m molecules are linked together, and in the single-chain dimer, the β2m molecule can or can not be membrane bound. In another aspect, the step of forming the immunogen of the present disclosure includes recombinantly expressing the MHC heavy chain and the MHC light chain separately in E. coli, and then refolding the MHC heavy and light chains with peptide in vitro.

Immunogens provided herein can further be modified for providing better performance or for aiding in stabilization of the immunogen. Examples of modifications which can be utilized in accordance with the present disclosure include but are not limited to, modifying an anchor and/or tail in the MHC-peptide complex, modifying one or more amino acids in the MHC-peptide complex, PEGylation, chemical cross-linking, changes in pH or salt depending on the specific peptide of the MHC-peptide complex, addition of one or more chaperone proteins that stabilize certain MHC-peptide complexes, addition of one or more adjuvants that enhance immunogenicity (such as but not limited to the addition of a T cell epitope on a multimer), combinations thereof, and the like.

MHC-Peptide Complex Screening http://patft.uspto.gov/netacgi/nph-Parser? Sect1=PTO1&Sect2=HITOFF&d=PALL&p=1&u=%2Fnetahtm1%2FPTO%2Fsrchnu m.htm&r=1&f=G&1=50&s1=5,635,363.PN.&OS=PN/5,635,363&RS=PN/5,635,363-h0#h0http://patft.uspto.gov/netacgi/nph-Parser? Sect1=PTO1 &Sect2=HITOFF&d=PALL&p=1&u=%2Fnetahtm1%2FPTO%2Fsrchnu m.htm&r=1&f=G&1=50&s1=5,635,363.PN.&OS=PN/5,635,363&RS=PN/5,635,363-h2#h2

Proper folding of HLA-peptide complexes can be determined as described in EXAMPLE 1 by forming a HLA-peptide complex tetramer and testing its capacity to bind HLA-peptide specific CD8+ T cells.

Soluble tetramers of major histocompatibility complex (MHC) proteins, charged with specific peptide antigen and fluorescently labeled (i.e., “MHC tetramers”), have proven useful in the detection, enumeration, characterization and purification of antigen-specific T lymphocytes. Applications of MHC class I tetramers have recently been reviewed in Doherty et al., Annu Rev Immunol, 18:561-592 (2000); Ogg et al., Immunol Lett, 66(1-3):77-80 (1999); Maini et al., Immunol Today, 20(6):262-266 (1999); and Doherty, Curr Opin Microbiol, 1(4):419-422 (1998). Applications of MHC class II tetramers are discussed in Reichstetter et al., J Immunol, 165(12):6994-6998 (2000); Kwok et al., J Immunol, 164(8):4244-4249 (2000); Liu et al., Proc Natl Acad Sci USA, 97(26):14596-14601 (2000); Novak et al., J Clin Invest, 104(12):R63-67 (1999); Crawford et al., Immunity, 8:675-682 (1998); and Kozono et al., Nature, 369:151-154 (1994). MHC tetramers have been shown to bind to T cell receptors (“TCR”) of T lymphocytes in an antigen- and MHC-specific manner (see e.g., Altman et al., Science, 274:94 (1996) and U.S. Pat. No. 5,635,363).

For multimerizing two or more copies of a MHC-peptide complex, each of the MHC-peptide complexes can be modified in some manner known in the art to enable attachment of the MHC-peptide complexes to each other, or the multimer can be formed around a substrate to which each copy of the MHC-peptide complex is attached. A tail can be attached to the two or more MHC-peptide complexes to aid in multimerization, wherein the tail can be selected from the group including but not limited to, a biotinylation signal peptide tail, an immunoglobulin heavy chain tail, a TNF tail, an IgM tail, a Fos/Jun tail, and combinations thereof. The multimer can contain any desired number of MHC-peptide complexes and thus, form any multimer desired, such as but not limited to, a dimer, a trimer, a tetramer, a pentamer, a hexamer, and the like. Streptavidin has four binding sites for biotin, so a BSP (biotinylation signal peptide) tail can be attached to the MHC molecule during production thereof, and a tetramer of the desired MHC-peptide complex could be formed by combining the MHC-peptide complexes with the BSP tails with biotin added enzymatically in vitro. An immunoglobulin heavy chain tail can be utilized as a substrate for forming a dimer, while a TNF tail can be utilized as a substrate for forming a trimer. An IgM tail could be utilized as a substrate for forming various combinations, such as tetramers, hexamers and pentamers. In addition, the MHC-peptide complexes can be multimerized through liposome encapsulation or artificial antigen presenting cell technology (see U.S. patent application Ser. No. 10/050,231, filed by Hildebrand et al. on Jan. 16, 2002, the contents of which are hereby expressly incorporated herein by reference). Further, the MHC-peptide complexes can be multimerized through the use of polymerized streptavidin and would produce what is termed a “streptamer”.

The MHC tetramer is rendered detectable by direct or indirect conjugation to a fluorophore. Typically, direct conjugation is accomplished by multimerizing the MHC-peptide molecules using a streptavidin or avidin molecule that has been prior-conjugated to a fluorophore. Such avidin and streptavidin proteins are commercially available from a variety of vendors (e.g., Becton Dickinson Immunocytometry Systems, San Jose, Calif., USA; Biomeda, Foster City, Calif., USA; Ancell Corp., Bayport, Minn. USA; Southern Biotechnology Assocs., Inc., Birmingham, Ala., USA). Indirect labeling can be performed using a fluorophore-conjugated antibody having specificity for the avidin/streptavidin moiety or for nonpolymorphic determinants of the multimerized MHC chain.

### Host Immunization and Antibody Production

Once the immunogen is produced and stabilized, it is delivered to a host for eliciting an immune response. The host can be any animal known in the art that is useful in biotechnological screening assays and is capable of producing recoverable antibodies when administered an immunogen, such as but not limited to, rabbits, mice, rats, hamsters, monkeys, baboons and humans. In one aspect, the host is a mouse, such as a Balb/C mouse or a transgenic mouse. In another aspect, the mouse is transgenic for the particular MHC molecule of the immunogen so as to minimize the antigenicity of the immunogen, thereby ensuring that the 3-dimensional domain of the peptide sitting in the binding pocket of the MHC molecule is the focus of the antibodies generated thereto and thus is preferentially recognized with high specificity. In yet another aspect, the mouse is transgenic and produces human antibodies, thereby greatly easing the development work for creating a human therapeutic.

As used herein, the term “antibody” refers to any immunoglobulin or intact molecule as well as to fragments thereof that bind to a specific epitope. Such antibodies include, but are not limited to polyclonal, monoclonal, chimeric, humanized, single chain, Fab, Fab′, F(ab)′ fragments and/or F(v) portions of the whole antibody. For example, the antibody “anti-LMP-1 monoclonal” used interchangeably herein with “anti HLA-A201/LMP-1”, and “anti HLA-A201/LMP-1 monoclonal” is capable of specifically binding to at least one EBV derived peptide of the present disclosure, including but not limited to LMP-1 (SEQ ID NO:7), or to a variant or fragment thereof, and includes monoclonal antibodies, polyclonal antibodies, single-chain antibodies, and fragments thereof which retain the antigen binding function of the parent antibody.

An intact “antibody” comprises at least two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH_{I}, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind. Examples of binding include (i) a Fab fragment, a monovalent fragment consisting of the V_{L}, V_{H}, C_{L} and CH1 domains; (ii) a F(ab)₂ fragment, a bivalent fragment comprising two Fab fragments linked by a disulfide bridge at the hinge region; (iii) a Fd fragment consisting of the VH and CH1 domains; (iv) a Fv fragment consisting of the V_{L} and V_{H} domains of a single arm of an antibody, (v) a dAb fragment (Ward et al., Nature, 341:544-546 (1989)), which consists of a VH domain; and (vi) an isolated complementarity determining region (CDR).

As used herein, the term “single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single Protein chain in which the V_{L} and V_{H} regions pair to form monovalent molecules (known as single chain Fv (scFv); see, e.g., Bird et al., Science, 242:423-426 (1988); and Huston et al., Proc Natl Acad Sci USA, 85:5879-5883 (1988)). Such single chain antibodies are included by reference to the term “antibody” fragments can be prepared by recombinant techniques or enzymatic or chemical cleavage of intact antibodies.

As used herein, the term “human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT App. Pub. Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins can be produced. See, Cabilly, U.S. Pat. No. 4,816,567, incorporated herein by reference in its entirety and for all purposes; and Queen et al., Proc Natl Acad Sci USA, 86:10029-10033 (1989).

As used herein, the term “monoclonal antibody” refers to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one aspect, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

As used herein, the term “immune cell response” refers to the response of immune system cells to external or internal stimuli (e.g., antigen, cell surface receptors, cytokines, chemokines, and other cells) producing biochemical changes in the immune cells that result in immune cell migration, killing of target cells, phagocytosis, production of antibodies, other soluble effectors of the immune response, and the like.

As used herein, the term “antibody fragment” refers to an incomplete or isolated portion of the full sequence of the antibody which retains the antigen binding function of the parent antibody. Examples of antibody fragments include Fab, Fab′, F(ab′)2, and Fv fragments; diabodies; linear antibodies; single-chain antibody molecules; and multispecific antibodies formed from antibody fragments. Fragments of the LMP-1 antibodies are encompassed by the invention so long as they retain the desired affinity of the full-length antibody. In particular, it can be shorter by at least one amino acid. For example, the fragment of an LMP-1 antibody comprises the antigen binding function that enables it to bind to LMP-1 (SEQ ID NO:7), or to a variant or fragment thereof.

As used herein, the term “antigen” refers to a substance that prompts the generation of antibodies and can cause an immune response. It can be used interchangeably in the present disclosure with the term “immunogen”. In the strict sense, immunogens are those substances that elicit a response from the immune system, whereas antigens are defined as substances that bind to specific antibodies. An antigen or fragment thereof can be a molecule (i.e., an epitope) that makes contact with a particular antibody. When a protein or a fragment of a protein is used to immunize a host animal, numerous regions of the protein can induce the production of antibodies (i.e., elicit the immune response), which bind specifically to the antigen (given regions or three-dimensional structures on the protein). The antigen can include but is not limited to LMP-1 and fragments thereof.

As used herein, the term “humanized antibody,” refers to at least one antibody molecule in which the amino acid sequence in the non-antigen binding regions has been altered so that the antibody more closely resembles a human antibody, and still retains its original binding ability.

Examples of the methods used for the production of the monoclonal antibodies of the present disclosure are given in EXAMPLES 2-5. These examples provide a method used for the production of monoclonal antibody specific for a peptide of SEQ ID NO:7 derived from the tumor virus antigen, latent membrane protein 1 (LMP-1) from EBV using Balb/C mice.

In addition, techniques developed for the production of “chimeric antibodies” (Morrison, et al., Proc Natl Acad Sci, 81:6851-6855 (1984), incorporated herein by reference in their entirety) by splicing the genes from a mouse antibody molecule of appropriate antigen specificity together with genes from a human antibody molecule of appropriate biological activity can be used. For example, the genes from a mouse antibody molecule specific for an autoinducer can be spliced together with genes from a human antibody molecule of appropriate biological activity. A chimeric antibody is a molecule in which different portions are derived from different animal species, such as those having a variable region derived from a murine mAb and a human immunoglobulin constant region.

In addition, techniques have been developed for the production of humanized antibodies (see, e.g., U.S. Pat. No. 5,585,089 and U.S. Pat. No. 5,225,539, which are incorporated herein by reference in their entirety). An immunoglobulin light or heavy chain variable region consists of a “framework” region interrupted by three hypervariable regions, referred to as complementarity determining regions (CDRs). Briefly, humanized antibodies are antibody molecules from non-human species having one or more CDRs from the non-human species and a framework region from a human immunoglobulin molecule.

Alternatively, techniques described for the production of single chain antibodies can be adapted to produce single chain antibodies against an immunogenic conjugate of the present disclosure. Single chain antibodies are formed by linking the heavy and light chain fragments of the Fv region via an amino acid bridge, resulting in a single chain polypeptide. Fab and F(ab′)2 portions of antibody molecules can be prepared by the proteolytic reaction of papain and pepsin, respectively, on substantially intact antibody molecules by methods that are well-known. See e.g., U.S. Pat. No. 4,342,566. Fab′ antibody molecule portions are also well-known and are produced from F(ab′)2 portions followed by reduction of the disulfide bonds linking the two heavy chain portions as with mercaptoethanol, and followed by alkylation of the resulting protein mercaptan with a reagent such as iodoacetamide.

In particular, the antibody, or fragment thereof, is capable of specifically binding to at least one EBV derived peptide. The new monoclonal antibody is capable of targeting EBV associated tumor cells based on their surface expression of EBV derived peptides in association with human MHC class I (specifically on a form termed human leukocyte antigen-HLA-A201).

The antibodies of the present disclosure can bind to the HLA-LMP-1 peptide monomers on the surface of EBV infected cells and target them for immune mediated lysis. This type of monoclonal antibody has a high affinity for HLA-LMP-1 peptide monomers, can recognize EBV-infected human B-lymphocytes, can bind to EBV-infected human tumor cell lines and can target EBV-infected tumor cells for antibody dependent cellular cytotoxicity (ADCC) by natural killer cells.

According to certain aspects, the present disclosure provides a TCR-like antibody produced according to the method comprising the steps of: forming an immunogen comprising a monomeric MHC-peptide complex; administering an effective amount of the immunogen to a host for eliciting an immune response to the peptide within the MHC-peptide complex; selecting a B cell specific to the peptide in the MHC-peptide complex; forming a hybridoma by fusing the B cell with an immortalized cell; and isolating an antibody produced by the hybridoma.

According to further aspects, the present disclosure provides a TCR-like antibody, wherein the antibody is selected from the group consisting of an antibody produced by a hybridoma cell line having ATCC Accession No. PTA-10351; an antibody having the binding characteristics of the antibody produced by the hybridoma cell line having ATCC Accession No. PTA-10351; an antibody that binds to an epitope capable of binding the antibody produced by the hybridoma cell line having ATCC Accession No. PTA-10351; an antibody that binds to an epitope comprising the amino acid sequence of SEQ ID NO:7, a variant, mutant, or fragment thereof; and an antibody comprising at least one light chain and at least one heavy chain, wherein the light chain comprises an amino acid sequence selected from the group consisting of SEQ ID NOS:1 to 3, a variant, mutant or fragment thereof and the heavy chain comprises an amino acid sequence selected from the group consisting of SEQ ID NOS:4 to 6, a variant, mutant or fragment thereof.

According to certain aspects, the present disclosure provides methods for producing a T cell receptor-like antibody, the method comprising the steps of: forming an immunogen comprising a monomeric MHC-peptide complex; administering an effective amount of the immunogen to a host for eliciting an immune response to the peptide within the MHC-peptide complex; selecting a B cell specific to the peptide in the MHC-peptide complex; forming a hybridoma by fusing the B cell with an immortalized cell; and isolating an antibody produced by the hybridoma.

According to further aspects, the present disclosure provides for methods of selecting a B cell, the method comprising the steps of incubating a B cell specific to the peptide within the MHC-peptide complex with a biotinylated MHC-peptide complex, wherein the MHC-peptide complex is capable of binding the B cell; and binding the biotinylated MHC-peptide complex with an anti-biotin conjugated bead.

According to further aspects, the present disclosure provides for methods of producing a T cell receptor-like antibody, wherein the antibody is produced by the hybridoma cell line having ATCC Accession No. PTA-10351.

The antibody can be labeled with at least one radionuclide in order to improve targeting of EBV infected tumor cells in vivo in at least a diagnostic and/or therapeutic capacity. For example, detecting EBV infected nasopharyngeal carcinoma cells in disparate lymph nodes after the tumor has metastasized by positron emission tomography (PET). The antibody labeled with the radionuclide enables an otolaryngologist to better target the diseased lymph nodes for surgery and/or radiotherapy. The antibody can be labeled with at least one toxin and/or chemotherapeutic reagent. In particular, the labeled antibody can be used as an immunotoxin that better targets these toxic agents to tumor cells.

The TCR-like antibodies produced by the present methods are superior to earlier described TCR-like antibodies. In fact, the methods of the present disclosure are more robust, reproducible, and give rise to qualitatively improved antibodies with improved selectivity.

It will be appreciated that once the CDRs of an antibody are identified, conventional genetic engineering techniques can be used to devise expressible polynucleotides encoding any of the forms or fragments of antibodies described herein.

After the host is immunized and allowed to elicit an immune response to the immunogen, a screening assay can be performed to determine if the desired antibodies are being produced. In certain aspects, the antibody specificity is determined based on binding to: (A) properly folded HLA trimer complexes (a HLA heavy chain, β2m, and peptide) containing an irrelevant peptide; (B) properly folded HLA monomers containing the peptide of interest; and (C) at least one antibody which recognizes mouse IgG and IgA constant regions and is covalently linked to a disclosing agent, such as but not limited to, peroxidase or alkaline phosphatase.

### B Cell Selection

Pre-purifying the antigen specific B cells prior to myeloma fusion from the immunized mice, using a biotinylated form of the monomer linked to an immunomagnetic bead, greatly improves the percentage of hybridomas that have the correct specificity compared to standard hybridoma approaches.

In another aspect, cell sorting is utilized to isolate desired B cells, such as B memory cells, prior to hybridoma formation. One method of sorting which can be utilized in accordance with the present disclosure is a sorting method using magnetic beads, such as those produced by Dynal or Miltenyi, can be utilized. Another method of B cell selection that can be used is fluorescence-activated cell sorting (FACS). Since B memory cells have immunoglobulin on their surface, this specificity can be utilized to identify and capture these cells. Optionally, beads can be coated with peptide/HLA complex and attached to a column. B cells with immunoglobulin on their surface can be identified by FACS as well as by binding to the complex.

In certain aspects, B cells are selected by first biotynylating HLA monomers/immunogens that bind to specific B cell receptors found on the surface of B cells specific to the monomer. Anti-biotin coated magnetic-activated cell sorting (MACs) beads can then be used to isolate bound B cells on magnetic columns.

In another aspect, the sorted B cells can further be differentiated and expanded into plasma cells, which secrete antibodies, screened for specificity and then used to create hybridomas or have their antibody genes cloned for expression in recombinant form.

Methods of Producing Hybridomas

According to another aspect, methods are provided for producing a hybridoma, the method comprising the steps of: forming an immunogen comprising a monomeric MHC-peptide complex; administering an effective amount of the immunogen to a host for eliciting an immune response to the peptide within the MHC-peptide complex; selecting a B cell specific to the peptide in the MHC-peptide complex; forming a hybridoma by fusing the B cell with an immortalized cell; and isolating the hybridoma.

According to further aspects, the present disclosure provides an isolated hybridoma cell line deposited with ATCC (10801 University Blvd., Manassas, Va. 20110) on Sep. 17, 2009 with ATCC Accession No. PTA-10351.

As used herein, the term “hybridoma” refers to cells that have been engineered to produce a desired antibody in large amounts. For example to produce at least one hybridoma, B cells are removed from the spleen of an animal that has been challenged with the relevant antigen and fused with at least one immortalized cell. This fusion is performed by making the cell membranes more permeable. The fused hybrid cells (called hybridomas), will multiply rapidly and indefinitely and will produce at least one antibody. An example of a hybridoma cell line produced according to the method of the present disclosure includes but is not limited to LMP1#226 which produces antibody anti-LMP-1 monoclonal.

As used herein, the term “immortalized cells” are also known as transformed cells, i.e., cells whose growth properties have been altered. This does not necessarily mean that these are “cancer” or “tumor” cells, i.e., able to form a tumor if introduced into an experimental animal, although in some cases they can do so. Immortalized cell lines include but are not limited to NS1, Jurkat, HeLa, T2 and multiple B-lymphocyte cell lines immortalized in vitro by EBV infection including RAJI, C1R.A2, CM304, CM392, CM800, CF801, CM803, CM936, CM960, BCL986, CF1007 and CM1081 and the like.

The novel methods of producing hybridomas provided herein allow for the development of TCR-like anti-HLA-peptide specific monoclonal antibodies, with a higher degree of reliability than has been previously possible.

The method according to the invention does not include a method for making an HBV-antibody produced by the hybridoma PTA-10167.

In particular, the hybridoma can be a monoclonal antibody-producing hybridoma cell. Optionally, the hybridoma cell can be cultured and further steps can be carried out including: isolating a monoclonal antibody that specifically binds to the antigen of interest, wherein the antigen was used to immunize the non-human animal.

For the preparation of at least one monoclonal antibody of the present disclosure, any technique that provides for the production of antibody molecules by continuous cell lines in culture can be used, provided a step of selection of B cell specific to the antigen is carried out before the fusion with the immortalized cell. For instance, B cells can be fused with immortal cells (e.g., myeloma cells) using polyethylene glycol (PEG) as described in EXAMPLE 4.

### Antibody Assays

The methods of the present disclosure further include the step of assaying the antibodies of interest to confirm their specificity and to determine whether those antibodies cross-react with other MHC molecules.

As used herein, the terms “specific binding” or “specifically binding” refer to the interaction between the MHC-peptide complexes and their corresponding antibodies. The interaction is dependent upon the presence of a particular structure of the protein recognized by the binding molecule (i.e., the antigen or epitope). In order for binding to be specific, it should involve antibody binding of the peptide only in the context of the MHC-peptide complex and not the peptide alone, MHC alone, or another MHC-peptide complex.

Once the antibodies are sorted, they are assayed to confirm that they are specific for one MHC-peptide complex and to determine whether they exhibit any cross reactivity with other HLA molecules. One method of conducting such assays is a sera screen assay as described in U.S. Patent App. Pub. No. 2004/0126829, the contents of which are hereby expressly incorporated herein by reference. However, other methods of assaying for quality control are within the skill of a person of ordinary skill in the art and therefore are also within the scope of the present disclosure.

Antibodies, or antigen-binding fragments, variants or derivatives thereof of the present disclosure can also be described or specified in terms of their binding affinity to a MHC-peptide complex. The affinity of an antibody for a MHC-peptide complex can be determined experimentally using any suitable method. (See, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-MHC-peptide complex interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and MHC-peptide complex, and a standardized buffer.

In some aspects, binding affinities range from 1×10⁸ to 1×10¹⁵ liter/mole, measured as an association constant. In further aspects, binding affinities range from 1×10⁹ to 1×10¹⁵ liter/mole. In further aspects, binding affinities range from 1×10¹⁰ to 1×10¹⁵ liter/mole. In further aspects, binding affinities range from 1×10¹¹ to 1×10¹⁵ liter/mole. In certain aspects, binding affinities are greater than 1×10⁸. In further aspects, binding affinity is greater than 1×10⁹ liter/mole. In certain preferred aspects, binding affinity is greater than 1×10¹⁰ liter/mole. In further aspects, binding affinity is greater than 1×10¹¹ liter/mole.

### Functional Moieties

In one aspect, the TCR-like antibody can have at least one functional moiety, such as but not limited to, a detectable moiety or a therapeutic moiety, bound thereto. For example but not by way of limitation, the detectable moiety can be selected from the group consisting of a fluorophore, an enzyme, a radioisotope and combinations thereof, while the therapeutic moiety can be selected from the group consisting of a cytotoxic moiety, a toxic moiety, a cytokine moiety, a bi-specific antibody moiety, and combinations thereof.

Many methods are known in the art to conjugate or fuse (couple) molecules of different types, including peptides. These methods can be used according to the present disclosure to couple an antibody another moiety, such as a therapeutic moiety or an identifiable moiety, to thereby provide an immunotoxin or immunolabel.

Two isolated peptides can be conjugated or fused using any conjugation method known to one skilled in the art. A peptide can be conjugated to an antibody of interest, using a 3-(2-pyridyldithio)propionic acid N-hydroxysuccinimide ester (also called N-succinimidyl 3-(2-pyridyldithio) propionate) (“SDPD”), a glutaraldehyde conjugation procedure, or a carbodiimide conjugation procedure.

Any SPDP conjugation method known to those skilled in the art can be used. For example, in one illustrative aspect, the method of conjugation by Cumber et al., Methods of Enzymology, 112:207-224 (1985), can be used and is incorporated herein by reference in its entirety.

Conjugation of a peptide (e.g., an identifiable or therapeutic moiety) with an antibody can be accomplished by methods known to those skilled in the art using glutaraldehyde. For example, in one illustrative aspect, the method of conjugation by G. T. Hermanson, “Antibody Modification and Conjugation, in Bioconjugate Techniques”, Academic Press, San Diego (1996) can be used and is incorporated herein by reference in its entirety.

Conjugation of a peptide with an antibody can be accomplished by methods known to those skilled in the art using a dehydrating agent such as a carbodiimide. Most preferably the carbodiimide is used in the presence of 4-dimethyl aminopyridine. As is well known to those skilled in the art, carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond).

Likewise, carbodiimide coupling can be used to form analogous covalent bonds between a carbon group of an antibody and a hydroxyl, amino or sulfhydryl group of the peptide. See, generally, J. March, Advanced Organic Chemistry: Reaction's, Mechanism, and Structure, 3d ed.:349-50 & 372-74 (1985). By means of illustration, and not limitation, the peptide is conjugated to an antibody via a covalent bond using a carbodiimide, such as dicyclohexylcarbodiimide. See generally, the methods of conjugation by B. Neises et al., Angew Chem, Int Ed Engl, 17:522 (1978); A. Hassner et al., Tetrahedron Lett, 4475 (1978); E. P. Boden et al., J Org Chem, 50:2394 (1986) and L. J. Mathias, Synthesis, 561 (1979). These references are incorporated herein by reference in their entirety.

### Methods of Using TCR-Like Antibodies

According to certain aspects, the present disclosure provides a method of detecting the presence of EBV, and/or at least one EBV infected cell in a subject, the method comprising: contacting at least one antibody according to any aspect of the present disclosure with at least one sample obtained from at least one subject; and detecting binding of the antibody to EBV and/or EBV infected cell.

In particular, the TCR-like antibody binding confirms that a cell can be EBV infected, that the sample comprises at least one EBV infected cell, and/or that the subject can be EBV positive. More in particular, the method can be an in vitro method of detection.

According to further aspects, the present disclosure provides a method of treatment of at least one EBV-linked disease, the method comprising administering to a subject in need thereof at least one TCR-like antibody or a fragment thereof.

The EBV-linked diseases can be lymphoproliferative disease, infectious mononucleosis, Nasopharyngeal Carcinoma, Burkitt's Lymphoma, B-cell non-Hodgkin Lymphoma, Hodgkin's Disease, Gastric Adenocarcinoma, extranodal T-/NK-cell Lymphoma, leiomyosarcoma, breast cancer and/or the like.

The TCR-like antibodies can be utilized in a variety of immunology related uses. In one aspect, the TCR-like antibodies can be utilized as direct therapeutic agents, either as an antibody or bispecific molecule. In another aspect, the TCR-like antibodies of the present disclosure can be utilized for viral profiling, to provide an individualized approach to EBV detection and treatment. The term “viral profiling” as used herein refers to the screening of cells with TCR-like antibodies of various specificities to define a set of MHC-peptide complexes on the virus infected cell. In another aspect, the TCR-like antibodies of the present disclosure could be utilized for vaccine validation, as a useful tool to determine whether desired T cell epitopes are displayed on cells such as but not limited to, tumor cells, virus infected cells, parasite infected cells, and the like. The TCR-like antibodies of the present disclosure could also be used as research reagents to understand the fate of antigen processing and presentation in vivo and in vitro, and these processes could be evaluated after exposure to a vaccine, and the like. The TCR-like antibodies of the present disclosure could also be utilized as vehicles for drug transport to transport payloads of toxic substances to virus infected cells or tumor cells. In addition, the TCR-like antibodies of the present disclosure could also be utilized in metabolic typing, such as but not limited to, to identify disease-induced modifications to antigen processing and presentation as well as HLA-peptide presentation and sensitivity to drugs.

In further aspects, the present disclosure provides for methods of mediating lysis of cells expressing at least one specific MHC-peptide complex on a surface thereof. The method includes providing a TCR-like antibody as described herein (wherein the TCR-like antibody is reactive against a specific MHC-peptide complex), and contacting the cells expressing at least one specific MHC-peptide complex on a surface thereof with the TCR-like antibody, such that the TCR-like antibody mediates lysis of the cells expressing the at least one specific MHC-peptide complex on a surface thereof.

### Therapeutic Compositions

According to further aspects of the present disclosure there is provided a pharmaceutical composition comprising a therapeutically effective amount of a TCR-like antibody, the antibody further comprising a therapeutic moiety being conjugated to the antibody. Preferably, the pharmaceutical composition further comprises a pharmaceutically acceptable carrier. The therapeutic moiety can be, for example, a cytotoxic moiety, a toxic moiety, a cytokine moiety and a bi-specific antibody moiety.

Compositions for use in accordance with the present disclosure can be formulated in a conventional manner using one or more physiologically acceptable carriers comprising excipients and auxiliaries which facilitate processing of a therapeutic composition into preparations which can be used pharmaceutically. These therapeutic compositions can be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

The therapeutic compositions are generally formulated as sterile, substantially isotonic and in full compliance with all Good Manufacturing Practice (GMP) regulations of the U.S. Food and Drug Administration. From the foregoing description, various modifications and changes in the compositions and methods will occur to those skilled in the art. All such modifications coming within the scope of the appended claims are intended to be included therein. Each recited range includes all combinations and sub-combinations of ranges, as well as specific numerals contained therein.

When a therapeutically effective amount of a composition of the present method is administered by e.g., intradermal, cutaneous or subcutaneous injection, the composition is preferably in the form of a pyrogen-free, parenterally acceptable aqueous solution. The preparation of such parenterally acceptable protein or polynucleotide solutions, having due regard to pH, isotonicity, stability, and the like, is within the skill in the art. A preferred composition should contain, in addition to protein or other active ingredients of the present disclosure, an isotonic vehicle such as sodium chloride injection, Ringer's injection, dextrose injection, dextrose and sodium chloride injection, lactated Ringer's injection, or other vehicle as known in the art. The composition of the present disclosure can also contain stabilizers, preservatives, buffers, antioxidants, or other additives known to those of skill in the art. The agents of the present disclosure can be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compositions can be formulated readily by combining the active compounds with pharmaceutically acceptable carriers well known in the art. Such carriers enable the compounds of the present disclosure to be formulated as tablets, pills, dragees, powders, capsules, liquids, solutions, gels, syrups, slurries, suspensions and the like, for oral ingestion by a patient to be treated.

Therapeutic compositions for parenteral administration include aqueous solutions of the compositions in water-soluble form. Optionally, the suspension can also contain suitable stabilizers or agents which increase the solubility of the compositions to allow for the preparation of highly concentrated solutions. Alternatively, the active ingredient can be in powder form for constitution with a suitable vehicle, e.g., sterile pyrogen-free water, before use.

In general, enteral dosage forms for the therapeutic delivery of polypeptides are less effective because in order for such a formulation to be efficacious, the peptide must be protected from the enzymatic environment of the gastrointestinal tract. Additionally, the polypeptide must be formulated such that it is readily absorbed by the epithelial cell barrier in sufficient concentrations to effect a therapeutic outcome. The polypeptides of the present method can be formulated with uptake or absorption enhancers to increase their efficacy. Such enhancers include for example, salicylate, glycocholate/linoleate, glycholate, aprotinin, bacitracin, SDS caprate and the like. An additional detailed discussion of oral formulations of peptides for therapeutic delivery is found in Fix, J Pharm Sci; 85(12):1282-1285 (1996), and Oliyai, et al., Ann Rev Pharmacol Toxicol; 32:521-544 (1993), this aspect of these two references is incorporated herein by reference.

In further compositions, proteins or other active ingredients of the present method can be combined with other antiviral or anti-tumor agents. It is within the skill of the art to optimize the therapeutic compositions comprising TCR-like antibodies and any secondary agent based on the present disclosure. Those of skill in the art will be able to modify and adjust these therapeutic compositions in order to treat EBV related diseases in a subject using TCR-like antibodies alone or in combination with a secondary therapy.

According to certain aspects, the present disclosure provides for a TCR-like antibody or fragment thereof of the present disclosure for use as medicament.

According to further aspects, the present disclosure provides at least one use of a TCR-like for the preparation of a medicament for treatment of at least one EBV-linked disease.

The EBV-linked diseases can be lymphoproliferative disease, infectious mononucleosis, Nasopharyngeal Carcinoma, Burkitt's Lymphoma, B-cell non-Hodgkin Lymphoma, Hodgkin's Disease, Gastric Adenocarcinoma, extranodal T-/NK-cell Lymphoma, leiomyosarcoma, breast cancer and/or the like.

The medicaments can be a pharmaceutical composition comprising at least one pharmaceutically acceptable excipient, diluent, carrier and/or adjuvant. Given that EBV infection has been linked to the development of several important forms of human cancer including nasopharyngeal carcinoma (NPC), Burkitt's lymphoma (BL), Hodgkin's lymphoma (HL), gastric carcinoma, T cell lymphomas, leiomyosarcoma, and breast cancer the antibody of the present disclosure can be used as a targeted delivery system for chemotherapeutic drugs, cytokines, pro-inflammatory mediators and toxins that will target the tumors based on their infection with EBV.

The antibodies of the present disclosure can be administered in combination with other similar antibodies targeting different EBV peptides in association with different HLA-types. Most humans express up to 25 different peptide/HLA combinations from EBV proteins on their infected cells. Accordingly, at least 1, 2, 5, 20 or 25 different antibodies of the present disclosure can be administered simultaneously, all of which can be EBV tumor specific. The tumor cells and viruses can thus be given no opportunity to adapt to this form of therapy.

### Kits

The invention provides kits comprising the compositions, e.g., nucleic acids, expression cassettes, vectors, cells, and/or antibodies (e.g., TCR-like antibodies) of the invention. The kits also can contain instructional material teaching the methodologies and uses of the invention, as described herein.

According to certain aspects, the present disclosure provides for a TCR-like antibody or fragment thereof for use in a kit.

According to further aspects, the present disclosure provides a kit for diagnosing at least one EBV-linked disease, the kit comprising a TCR-like antibody.

In particular, the TCR-like antibody binding confirms that a cell can be EBV infected, that the sample comprises at least one EBV infected cell, and/or that the subject can be EBV positive. More in particular, the method can be an in vitro method of detection.

### Nucleic Acids

According to another aspect, the present disclosure provides an isolated nucleic acid molecule encoding: at least one heavy chain of a TCR-like antibody or a fragment thereof, wherein the heavy chain comprises an amino acid sequence selected from the group consisting of SEQ ID NOS:4 to 6, a variant, mutant or fragment thereof; and/or at least one light chain of a TCR-like antibody or a fragment, wherein the light chain comprises an amino acid sequence selected from the group consisting of SEQ ID NOS:1 to 3, a variant, mutant or fragment thereof.

According to further aspects, the present disclosure provides an expression vector comprising the nucleic acid which encodes a TCR-like antibody and a host cell comprising the expression vector. In particular, the vectors can comprise, but are not limited to, lentiviral vectors, retroviral vectors, adenoviral vectors, adeno-associated virus vectors and Herpes Simplex Virus vectors. More in particular, retroviral vectors can be used for delivery of the constructs either in vitro, ex vivo or in vivo.

### Recombinant Nucleic Acid Techniques

The nucleic acids used to practice this invention, whether RNA, siRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams, J Am Chem Soc, 105:661 (1983); Belousov, Nucleic Acids Res, 25:3440-3444 (1997); Frenkel, Free Radic Biol Med, 19:373-380 (1995); Blommers, Biochemistry, 33:7886-7896 (1994); Narang, Meth Enzymol, 68:90 (1979); Brown, Meth Enzymol, 68:109 (1979); Beaucage, Tetra Lett, 22:1859 (1981); U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook and Russell, ed., MOLECULAR CLONING: A LABORATORY MANUAL (3rd ED.), Vols. 1-3, Cold Spring Harbor Laboratory (2001); CURRENT PROTOCOLS 1N MOLECULAR BIOLOGY; Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, adioimmunoassay (RIM), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat Genet, 15:333-335 (1997); yeast artificial chromosomes (YAC); bacterial artificial chromosomes (BAC); P1 artificial chromosomes, see, e.g., Woon, Genomics, 50:306-316 (1998); P1-derived vectors (PACs), see, e.g., Kern, Biotechniques, 23:120-124 (1997); cosmids, recombinant viruses, phages or plasmids.

The invention provides fusion proteins and nucleic acids encoding a TCR-like antibody. A TCR-like antibody can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Amgen Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry, 34:1787-1797 (1995); Dobeli, Protein Expr Purif, 12:404-414 (1998)). The histidine residues facilitate detection and purification while the enterokinase cleavage site provides a means for purifying the epitope from the remainder of the fusion protein. In one aspect, a nucleic acid encoding a polypeptide is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell Biol, 12:441-53 (1993).

### Transcriptional Control Elements

The nucleic acids, as aspects of the invention, can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

### Expression Vectors and Cloning Vehicles

Aspects of the invention provide expression vectors and cloning vehicles comprising nucleic acids of the invention, e.g., sequences encoding the proteins of the invention. Expression vectors and cloning vehicles can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other, vectors specific for specific hosts of interest (such as bacillus, Aspergillus and yeast). Vectors can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature, 328:731 (1987); Schneider, Protein Expr Purif, 6435:10 (1995); Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picomnaviridiae. Chimeric vectors can also be employed which exploit advantageous merits of each of the parent vector properties (See e.g., Feng, Nature Biotechnology, 15:866-870 (1997)). Such viral genomes can be modified by recombinant DNA techniques to include the nucleic acids of the invention; and can be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J Virol, 66:2731-2739 (1992); Johann, J Virol, 66:1635-1640 (1992). Adeno-associated virus (AAV)-based vectors can be used to adioimmun cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther, 3:957-964 (1996).

As used herein, the term “expression cassette” refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

As used herein, the term “vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

### Host Cells and Transformed Cells

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector can be glycosylated or can be non-glycosylated. Polypeptides of the invention can or can not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

### Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the flgK protein, rpoN protein, or fliA protein, or subsequences thereof.

Amplification methods include, e.g., polymerase chain reaction, PCR (PCR PROTOCOLS, A GUIDE TO METHODS AND APPLICATIONS, ed. Innis, Academic Press, N.Y. (1990) and PCR STRATEGIES, ed. Innis, Academic Press, Inc., N.Y., ligase chain reaction (LCR) (1995) (see, e.g., Wu, Genomics, 4:560 (1989); Landegren, Science, 241:1077 (1988); Barringer, Gene, 89:117 (1990)); transcription amplification (see, e.g., Kwoh, Proc Natl Acad Sci USA, 86:1173 (1989)); and, self-sustained sequence replication (see, e.g., Guatelli, Proc Natl Acad Sci USA, 87:1874 (1990)); Q Beta replicase amplification (see, e.g., Smith, J Clin Microbiol, 35:1477-1491 (1997)), automated Q-beta replicase amplification assay (see, e.g., Burg, Mol Cell Probes, 10:257-271 (1996)) and other RNA polymerase mediated techniques (e.g., NASBA, Cangene, Mississauga, Ontario); see also Berger, Methods Enzymol, 152:307-316 (1987); Sambrook; Ausubel; U.S. Pat. Nos. 4,683,195 and 4,683,202; Sooknanan, Biotechnology, 13:563-564 (1995).

### Hybridization of Nucleic Acids

The invention provides isolated or recombinant nucleic acids that hybridize under stringent conditions to an exemplary sequence of the invention. In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 350, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

As used herein, the term “selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one aspect, a nucleic acid can be determined to be within the scope of the invention by its ability to hybridize under stringent conditions to a nucleic acid otherwise determined to be within the scope of the invention (such as the exemplary sequences described herein).

As used herein, the term “stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook and Russell, ed., MOLECULAR CLONING: A LABORATORY MANUAL (3rd ED.), Vols. 1-3, Cold Spring Harbor Laboratory (2001); CURRENT PROTOCOLS IN MOLECULAR BIOLOGY; Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, PART I. Theory and Nucleic Acid Preparation, Tijssen, ed. Elsevier, N.Y. (1993).

Generally, stringent conditions are selected to be about 5-10° C. lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide as described in Sambrook (cited above). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present disclosure, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

### Determining the Degree of Sequence Identity

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2 or FASTA version 3.0t78 algorithms and the default parameters discussed below can be used.

As used herein, the term “comparison window” includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv Appl Math, 2:482 (1981), by the homology alignment algorithm of Needleman & Wunsch, J Mol Biol, 48:443 (1970), by the search for similarity method of Pearson & Lipman, Proc Natl Acad Sci USA, 85:2444 (1988), by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology, (1999 Suppl.)).

A preferred example of an algorithm that is suitable for determining percent sequence identity and sequence similarity is the FASTA algorithm, which is described in Pearson & Lipman, Proc Natl Acad Sci USA, 85:2444 (1988). See also Pearson, Methods Enzymol, 266:227-258 (1996). Preferred parameters used in a FASTA alignment of DNA sequences to calculate percent identity are optimized, BL50 Matrix 15: −5, k-tuple=2; joining penalty=40, optimization=28; gap penalty −12, gap length penalty=−2; and width=16.

Another preferred example of algorithm that is suitable for determining percent sequence identity and sequence similarity are the BLAST and BLAST 2.0 algorithms, which are described in Altschul et al., Nuc Acids Res, 25:3389-3402 (1977); and Altschul et al., J Mol Biol, 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc Natl Acad Sci USA, 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc Natl Acad Sci USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J Mol Evol, 35:351-360 (1987). The method used is similar to the method described by Higgins & Sharp, CABIOS, 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc Acids Res, 12:387-395 (1984)).

Another preferred example of an algorithm that is suitable for multiple DNA and amino acid sequence alignments is the CLUSTALW program (Thompson et al., Nucl Acids Res, 22:4673-4680 (1994)). ClustalW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff & Henikoff, Proc Natl Acad Sci USA, 89:10915-10919 (1992)).

As used herein, the term “sequence identity” refers to a measure of similarity between amino acid or nucleotide sequences, and can be measured using methods known in the art, such as those described below:

As used herein, the terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

As used herein, the term “substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred aspect, the sequences are substantially identical over the entire length of the coding regions.

As used herein, the terms “homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continuous residues ranging anywhere from 20 to the full length of an exemplary polypeptide or nucleic acid sequence of the invention are compared to a reference sequence of the same number of contiguous positions after the two sequences are optimally aligned.

The following examples of specific aspects for carrying out the present disclosure are offered for illustrative purposes only, and are not intended to limit the scope of the present disclosure in any way.

### EXEMPLARY ASPECTS

### Example 1

### Preparation of Antigen/Monomer

The EBV antigen targeted was latent membrane protein-1 (LMP-1) of YLLEMLWRL (SEQ ID NO:7). The monomer prepared was designed, expressed and an FPLC purified recombinant, membrane free, fully folded, heterotrimeric complex of at least one HLA-A201 heavy and light chain plus an antigenic peptide of SEQ ID NO:7.

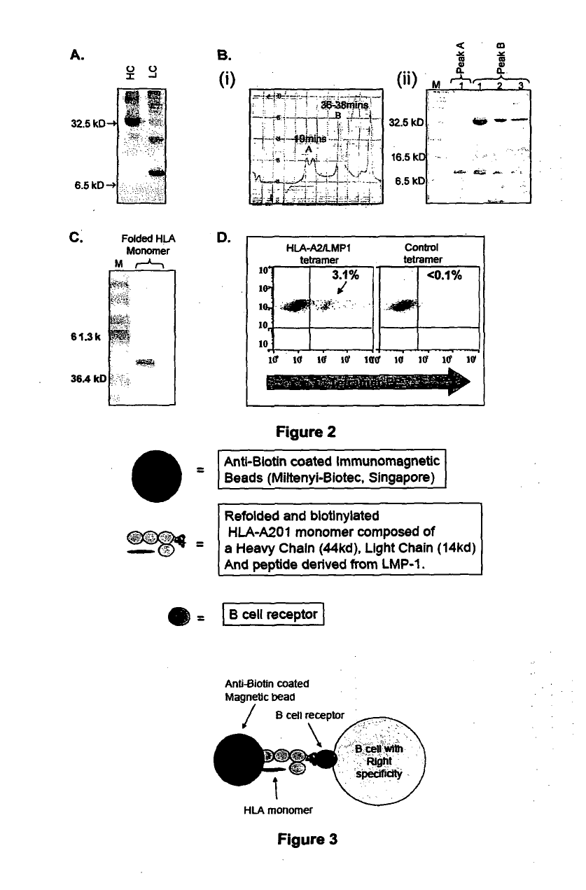

Human major histocompatibility complex (MHC) class-1HLA-A201 heavy chains (HC) and light chains (LC) (B2 microglobulin) were expressed as recombinant proteins in BL21 E. coli (Invitrogen, Singapore). The heavy and light chains were isolated as inclusion bodies and dissolved in 8M urea. FIG. 2(A) shows the result of an SDS-PAGE where the protein content of HC and LC inclusion bodies were analyzed.

The antigenic peptide of SEQ NO:7 from LMP-1 of EBV was then selected and refolded with the HLA heavy and light chains to form HLA-LMP-1 peptide monomers in vitro. Anion-exchange chromatography was used to purify the monomers. FIG. 2(B) shows the results of the (i) fast protein liquid chromatography (FPLC) profile and (ii) SDS-PAGE analysis of the purified folded monomers. Peak A (fraction A) at 19 minutes contained light chain (β2m) only whereas peak B (fraction B) at 36-37 minutes contained both heavy chain (35kD) and light chain (12 kD).

Confirmation that fraction B from the FPLC results above contained fully folded HLA-LMP-1 peptide monomers was done using gel blots with a non-denaturing native gel where, fractions 1 and 2 collected from peak B were pooled and analyzed. The gel was immunoblotted with the anti-HLA conformation specific monoclonal w6/32 (BD-Pharmingen, Singapore). The results in FIG. 2(C) confirmed that the purified monomer was correctly folded.

HLA-LMP-1 peptide monomers were then tetramerized and tested for their capacity to bind LMP-1 specific CD8+ T cells. FIG. 2(D) shows that the tetramer bound to the CD8+ T cells from an EBV positive donor (left panel) versus a control tetramer (right panel). This confirmed that the HLA-LMP-1 peptide monomers had folded correctly prior to their use for immunizing mice.

### Example 2

### Immunization of a Mouse With HLA-LMP-1 Peptide Monomers

The HLA-LMP-1 peptide monomers from EXAMPLE 1 were used as a source of antigen to stimulate antibody responses in immunized Balb/C mice. Female Balb/C mice were immunized intraperitoneally with 25 μg of purified HLA-peptide monomers in 100 μl of Complete Freunds adjuvant (Sigma-Aldrich, Singapore). Mice were later boosted intraperitoneally with 25 μg of the same monomer in Incomplete Freunds (Sigma-Aldrich, Singapore) at days 21 and 35. A final intravenous boost via the tale vein of 25 μg of the same monomer in sterile saline was given at day 42. The mice were then euthanized by CO₂ asphyxiation at day 45 and their spleens aseptically removed.

### Example 3

### Selection of B Cells Specific to LMP-1 in the Context of the HLA-LMP-1 Complex

At day 45, splenocytes were prepared by gentle homogenization of the mouse spleens through a 70 μm sterile cell strainer (Invitrogen, Singapore) and washed twice in 20 ml of phosphate buffered saline solution (PBS) (Invitrogen Singapore). Splenocytes were pelleted in 50 ml polystyrene conical tubes (Invitrogen, Singapore) by centrifugation at 300 G for 7 minutes at 4° C. B cells with the desired specificity were purified as follows: splenocytes were incubated with 20 μg of biotinylated HLA-LMP-1 peptide monomers for 1 hour at 4° C. in 500 μl of sterile PBS in 50 ml polystyrene conical tubes. Unbound monomers were removed by washing the splenocytes in 20 ml of sterile PBS. The biotinylated monomers bound specific B cell receptors found on the surface of the B cell specific to the monomer and have the same specificity as the antibody that the B cell secretes. Anti-biotin conjugated immuno-magnetic beads (Miltenyi-Biotec, Singapore) were used at a concentration of 20 μl bead suspension per 1×10⁷ splenocyes to positively select the bound B-cells through binding to the biotinylated monomers on the B cell surface. Unbound beads were removed by washing the splenocytes twice in 5 ml of sterile PBS. The B cells were then enriched on magnetic columns (Miltenyi-Biotec, Singapore) via immunomagenetic selection (FIG. 3). Non-specific cells were removed by rinsing the columns four times in 3 ml of sterile PBS.

Pre-purifying the antigen specific B cells prior to myeloma fusion from the immunized mice, using a biotinylated form of the monomer linked to an immunomagnetic bead greatly improved the percentage of hybridomas that have the correct specificity compared to standard hybridoma approaches (FIG. 5).

### Example 4

### Fusion of Selected B Cell with Immortalized Cell

Selected B cells from EXAMPLE 3 were fused with NS1 myeloma cells using polyethylene glycol (PEG). Myeloma cells were pipetted into 96-well microlitre plates with peritoneal macrophage feeder layers in hypoxanthine aminopterin thymidine (HAT) medium (Invitrogen, Singapore). Positive hybridoma colonies were scored from day 17 to day 28. Supernatants from hybridoma colonies were screened by flow cytometry (i.e., fluorescence activated cell sorting (FACS)) on HLA-A201/peptide positive cell lines. Screening with HLA-A201/peptide positive cell lines requires greater specificity than ELISA screening methods and thus, enables selection of only those hybridomas yielding highly specific antibodies.

The monoclonal antibodies created according to the above EXAMPLES represent unique immunological targeting reagents that enable a thorough investigation of EBV-LMP-1 antigen processing/presentation during latency and tumorigenesis and the targeting of LMP-1 expressing EBV-associated tumor cells in situ.

### Example 5

### Selection of Monoclonal Hybridomas Producing HLA-A201/LMP-1 Specific Antibodies

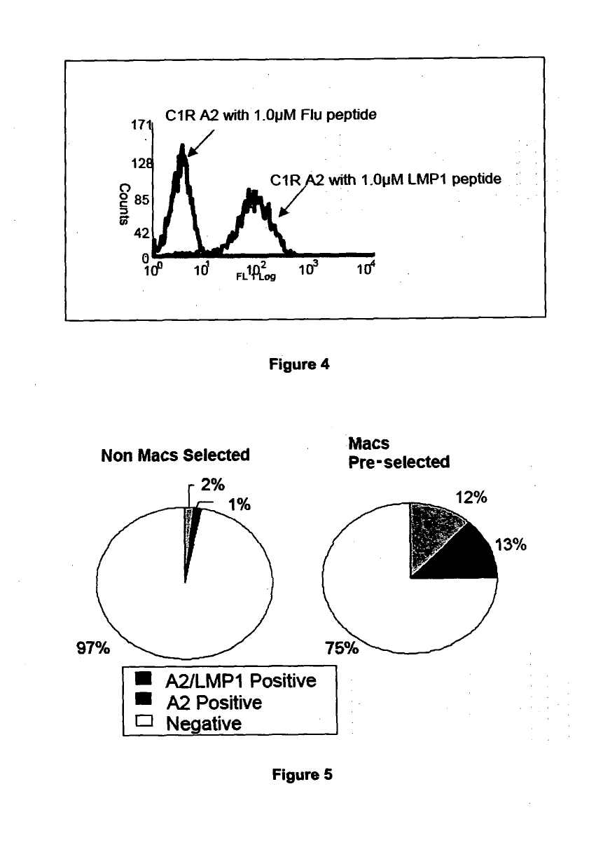

Flow cytometry was used to compare the binding affinities of antibodies in multiple hybridoma cell line supernatants for an HLA-A201 expressing human B cell line (C1R.A2) (FIG. 4, grey histogram), which was pulsed with the LMP-1 peptide (SEQ ID NO:7) or with an HLA-A201 restricted peptide from Influenza A (SEQ ID NO:8) (FIG. 4, black histogram). Hybridoma supernatants with clear specificity for the LMP-1 peptide on HLA-A201 were identified and selected using this methodology (FIG. 4). FIG. 4 shows the staining pattern of a B cell hybridoma clone LMP1#226, which yields TCR-like monoclonal antibodies with HLA-A201/LMP-1 specificity (grey histogram).

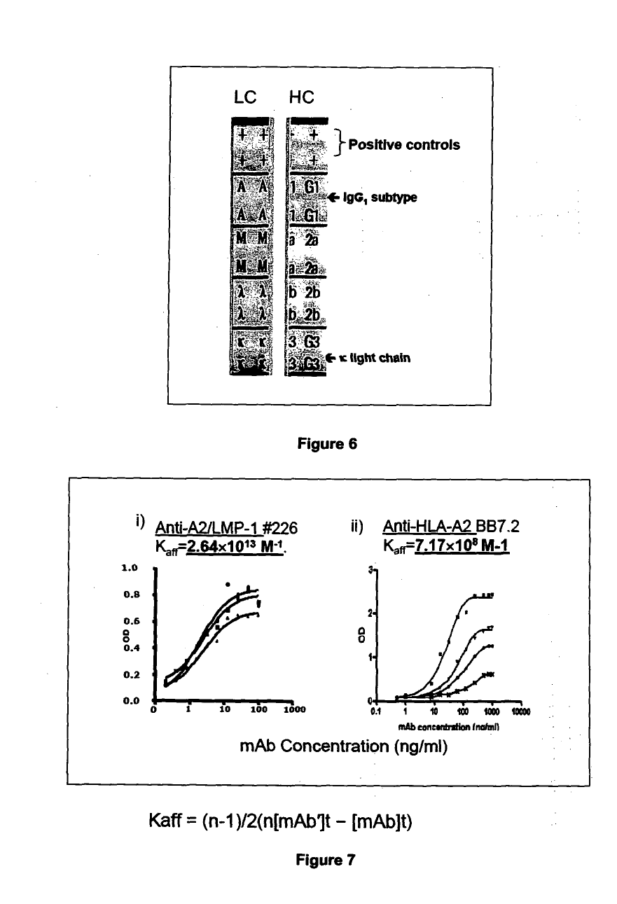

The immunoglobulin isotype test for anti HLA-A201/LMP-1 monoclonal antibodies for clone LMP1#226, as shown in FIG. 6, was identical to that seen for 24 other hybridoma clones with similar specificities. These results indicate that the clone LMP1#226 comprises a mouse IgG1 heavy chain and a Kappa light chain.

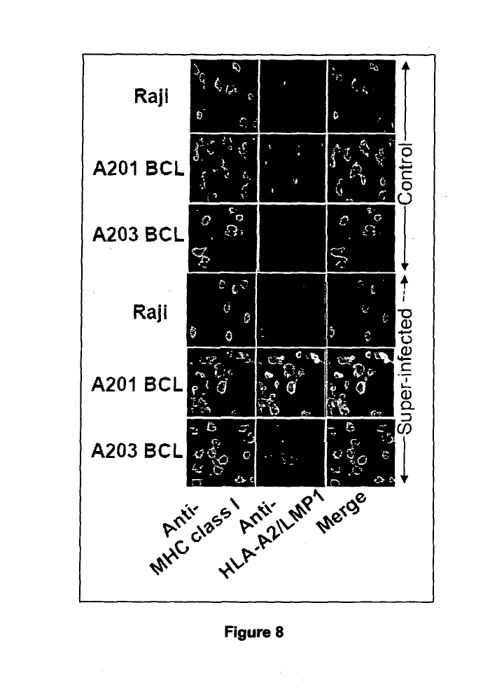

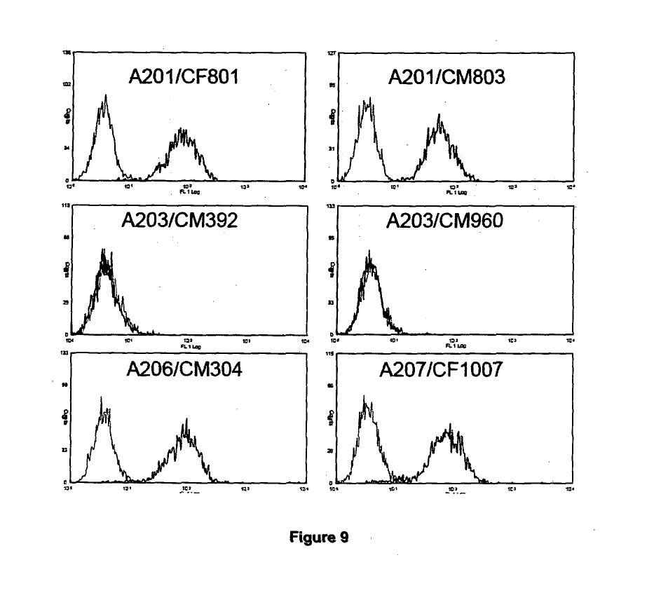

Confocal microscopy was used to determine whether clone LMP1#226 could detect human B lymphocyte cell lines naturally infected with EBV. The desired functionality was the ability to detect natural HLA-A201/LMP-1 peptide monomers generated during the course of an infection of human cells with EBV. HLA-A2 negative B cell lines (i.e., 2E and RAJI), HLA-A201 B cell line (i.e., CF986) and HLA-A203 B cell line (i.e., CF986) were super-infected with EBV for 24 hours prior to labeling with a polyclonal anti-HLA class-I and an antibody from LMP1#226 and analyzed by confocal microscopy. EBV infection of the HLA-A201 B cell line led to binding of the monoclonal antibody from LMP1#226 and the resulting staining pattern co-localized with the polyclonal anti-HLA. No significant binding was detected in the RAJI or HLA-A203 positive B cell line treated under similar conditions. Accordingly, it was shown that HLA-A201 positive human B-cells super-infected with high concentrations of EBV in vitro, could detect a significant degree of antibody binding compared to EBV negative infected control cell lines which were HLA-A201 negative (FIG. 8).

The ability of antibodies from clone LMP1#226 to detect HLA-LMP-1 peptide monomers in human cell lines expressing the Caucasian form of HLA-A2 (HLA-A201) versus the Asian variants of HLA-A2, HLA-A203, HLA-A206 and HLA-A207 was analyzed. The changes in amino acid sequence expressed by the different variants of HLA-A2 plus their frequency in Caucasian and Asian populations are illustrated in FIG. 9A. The ability of antibody from clone LMP1#226 to recognize these complexes by flow cytometry is shown in FIG. 9B. Cell lines, CF801 and CM803 (HLA-A201) showed a good degree of binding along with cell lines, CM304 and CF1007 (HLA-A206 and HLA-A207 respectively). Cell lines, CM392 and CM960 (HLA-A203) did not exhibit any binding suggesting that these HLA-A2 polymorphisms result in different epitope specificities.

### Example 6

### LMP-1 Detection in Latently Infected B Cells

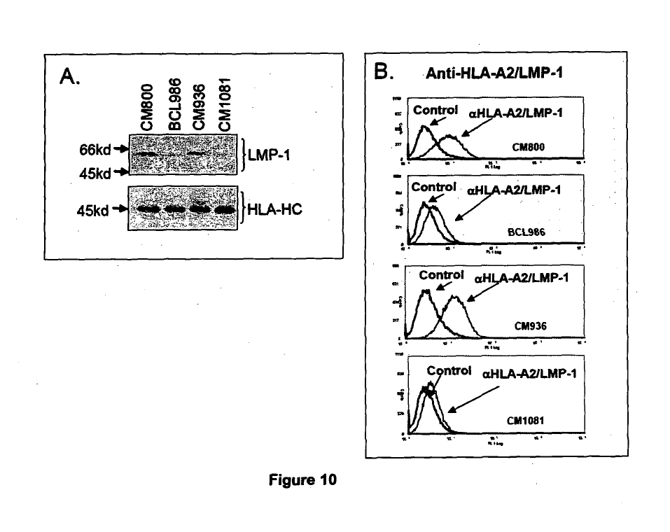

LMP-1 expression was detected in latently infected human B cells. Latent infection of EBV is normally associated with tumor cells. Under these circumstances, EBV gene expression is restricted to a small subset of genes including LMP-1. 1×10⁶ EBV transformed B cells were screened for the presence of LMP-1 using a commercially available anti-LMP-1 antibody (Acc Chem & Sci Co, USA) (top panel of FIG. 10(A)). Four human latently EBV-infected B cell lines that have been maintained in culture for up to 10 years, were studied for LMP-1 expression. Out of the four cell lines, LMP-1 expression in 50%, i.e., 2 out of the 4 cell lines were shown (FIG. 10(A)). A monoclonal antibody specific for human HLA (HLA-HC) was used as a loading control.

A flow cytometry based detection of surface HLA-LMP-1 peptide monomers on latent EBV infected B cell lines was carried out where the EBV immortalized human B cell lines were stained with 0.5 μg of anti-HLA-A201/LMP-1 at 40° C. for 20 minutes. Cells Were washed twice in ice cold phosphate buffered saline prior to incubation with 0.5 μg of goat-anti-mouse IgG-Alexa-Fluor 488 (Jackson Labs, USA) at 4° C. for 20 minutes. Stained cells were washed twice in ice cold phosphate buffered saline, fixed with 500 μl of 1% paraformaldehyde and analyzed on a Becton-Dickinson FACs Calibur Flow Cytometer. The results as shown in FIG. 10(B) revealed that the latently infected human B cell lines that expressed LMP-1 showed a small but significant shift in surface staining with antibody from clone LMP1#226, suggesting that this monoclonal antibody can be utilized to detect latent levels of LMP-1 such as those found in EBV associated tumor cells.

### Example 7

### Evaluating Overlapping Specificity Between TCR-Like Antibodies and TCRs

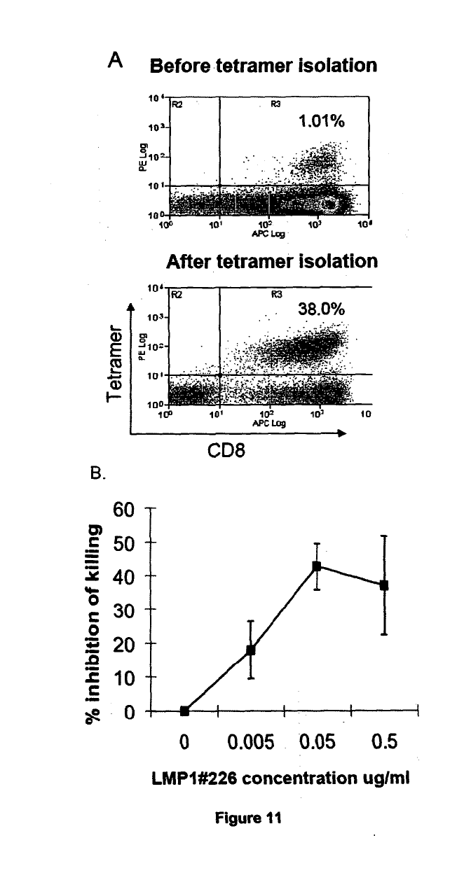

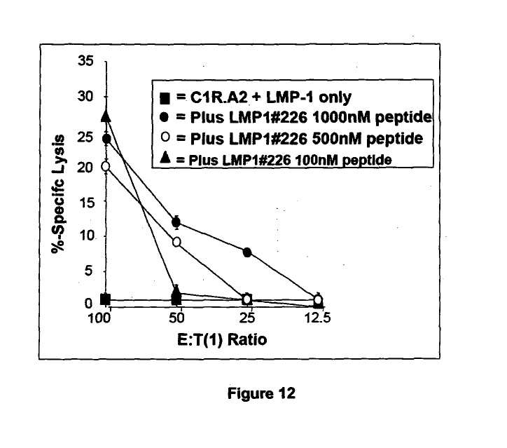

This EXAMPLE describes the characterization of overlapping specificities between the TCR-like antibody from clone LMP1#226 and the TCR employed by cytotoxic T cells for the LMP-1 peptide in association with HLA-A201. A human CD8+ cytotoxic T cell line specific for the LMP-1 peptide, with SEQ ID NO:7 in association with HLA-A201, was generated from a healthy human. This cell line was employed in a ⁵¹Cr release assay as described in MacAry et al., Immunity, 20:95-106 (2004) to assay cytotoxic killing of cellular targets expressing the LMP-1 peptide on HLA-A201. Briefly, target cells were labeled with 1 mCi of ⁵¹Cr (sodium chromate) (GE-Healthcare, Singapore) for 1 hour and were then washed twice in RPMI-1640 prior to being employed as targets in our assay. The target cells were pulsed with 5 mM LMP-1 peptide (SEQ ID NO:7) for 20 minutes at 37° C. Controls were not pulsed with the antigenic peptide. When the target cells were pre-incubated with antibody from clone LMP1#226, the degree of cytotoxic killing by the CD8+ T cells was reduced, as indicated by increased levels of ⁵¹Cr in the culture supernatants (TopCount, Beckman Coulter, Singapore). Diminished binding by the TCR suggests an overlap in the HLA-peptide complexes recognized by the T cells and antibody from clone LMP1#226 (FIG. 11).

### Example 8

### Determining Binding Affinity Constants

The affinity binding constant (K_{aff}) of clone LMP1#226 was determined using the following formula:

$${K}_{aff} = \frac{( n - 1 )}{2 ( {n [ {mAb}^{′} ]}_{t} - {[ mAb ]}_{t} )}$$

$$in which \text{:}$$

$$n = \frac{{[ mAg ]}_{t}}{{[ {mAg}^{′} ]}_{t}}$$

[mAb] is the concentration of free antigen sites, and [mAg] is the concentration of free monoclonal binding sites as determined at two different antigen concentrations (i.e., [mAg]ₜ and [mAg′]ₜ) (Beatty et al., J Imm Meth, 100:173-179 (1987)).

FIG. 7 shows the experimental dose response curve for TCR-like monoclonal antibodies (i) LMP1#226 and (ii) BB7.2 (anti-HLA-A2) at increasing concentrations of antigen. Antigen concentrations ranged from 7.5-100 ng/ml. The data points as shown in Tables 1 and 2 were fitted using four-parameter logistics. The summed square deviations were no higher than 0.393 for all of the data points expressed. Clone LMP1#226 was shown to have a high binding affinity (K_{aff}=2.16×10¹³) for HLA-A201/LMP-1 monomers.

TABLE 1
K_{aff} values for LMP1#226

| [Ag] | [mAb] at OD₅₀ | K_{aff} | Average K_{aff} |
|---|---|---|---|
| (ng/ml) | (ng/ml) | (×10¹³M⁻¹) | (M⁻¹) |
| 100 | 18.59 | 2.34 | 2.64 ± 0.28 × 10¹³ |
| 50 | 25.31 | 2.90 | |
| 25 | 25.60 | 2.68 | |

TABLE 2
K_{aff} values for BB7.2 (pan anti-HLA-A2). BB7.2 is a
commercially available anti-HLA-A2 antibody.

| [Ag] | [mAb] at OD₅₀ | K_{aff} | Average K_{aff} |
|---|---|---|---|
| (ng/ml) | (ng/ml) | (×10⁹M⁻¹) | (M⁻¹) |
| 120 | 26.102 | 0.57 | 7.17 ± 2.6 × 10⁸ |
| 60 | 54.117 | 1.01 | |
| 30 | 68.129 | 0.871 | |
| 15 | 121.15 | 0.431 | |

Antibodies from clone LMP1#226 were shown to be able to induce the NK cell mediated killing of EBV infected, transformed human B cells in a process termed antibody dependent cellular cytotoxicity (ADCC). Murine splenic NK cell effectors (based on their expression of NK1.1) were used with ⁵¹Cr labeled CIR.A2 cells as assay targets. The LMP-1 derived peptide was titrated onto the CIR.A2 target cells with unpulsed cells used as the negative control. After 6 hours, the number of human target cells killed was determined based on the quantity of ⁵¹Cr released into the culture supernatant. Results were expressed as percent specific lysis of LMP-1 peptide pulsed targets versus controls (mean of triplicate wells +/−SEM). Antibodies derived from clone LMP1#226 induced a significant degree of NK cell mediated killing of the human targets pulsed with LMP-1 peptide compared to controls.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications can be made thereto without departing from the spirit or scope of the appended claims.

What is claimed:

1. A method of producing a T cell receptor-like antibody, the method comprising the steps of
forming an immunogen comprising a monomeric MHC-peptide complex;
administering an effective amount of the immunogen to a host-for eliciting an immune response to the peptide within the MHC-peptide complex;
selecting a B cell specific to the peptide in the MHC-peptide complex;
forming a hybridoma by fusing the B cell with an immortalized cell; and
isolating an antibody produced by the hybridoma.

2. The method according to claim 1, wherein the step of selecting a B cell further comprises the steps of:
incubating a B cell specific to the peptide within the MHC-peptide complex with a biotinylated MHC-peptide complex, wherein the MHC-peptide complex is capable of binding the B cell; and
binding the biotinylated MHC-peptide complex with an anti-biotin conjugated bead.

3. The method of claim 1, wherein the antibody binds to the peptide in the MHC-peptide complex with an affinity of at least 1×10¹⁰ liter/mole, measured as an association constant (K_{aff}).

4. The method of claim 1, wherein the antibody is produced by the hybridoma cell line having ATCC Accession No. PTA-10351.

5. An isolated antibody or fragment thereof, produced according to the method of claim 1.

6. The antibody of claim 5, wherein the antibody binds to the peptide in the MHC-peptide complex with an affinity of at least 1×10¹⁰ liter/mole, measured as an association constant (K_{aff}).

7. The antibody according to claim 5, wherein the antibody is selected from the group consisting of
an antibody produced by a hybridoma cell line having ATCC Accession No. PTA-10351;
an antibody having the binding characteristics of the antibody produced by the hybridoma cell line having ATCC Accession No. PTA-10351;
an antibody that binds to an epitope capable of binding the antibody produced by the hybridoma cell line having ATCC Accession No. PTA-10351;
an antibody that binds to an epitope comprising the amino acid sequence of SEQ ID NO:7, a variant, mutant, or fragment thereof; and
an antibody comprising at least one light chain and at least one heavy chain, wherein the light chain comprises an amino acid sequence selected from the group consisting of SEQ ID NOS:1 to 3, a variant, mutant or fragment thereof and the heavy chain comprises an amino acid sequence selected from the group consisting of SEQ ID NOS:4 to 6, a variant, mutant or fragment thereof.

8. The antibody of claim 5, wherein the peptide is an EBV derived peptide.

9. The antibody of claim 5, wherein the EBV derived peptide is LMP-1, a variant, mutant, or fragment thereof.

10. The antibody of claim 5, wherein the peptide comprises SEQ ID NO:7.

11. The antibody of claim 5, wherein the antibody is a human, humanized, or chimeric antibody.

12. The antibody according to claim 5, wherein the antibody is labeled with a radionuclide.

13. The antibody according to claim 5, wherein the antibody is labeled with a toxin and/or chemotherapeutic reagent.

14. A method of producing a hybridoma, the method comprising the steps of
forming an immunogen comprising a monomeric MHC-peptide complex;
administering an effective amount of the immunogen to a host for eliciting an immune response to the peptide within the MHC-peptide complex;
selecting a B cell specific to the peptide in the MHC-peptide complex;
forming a hybridoma by fusing the B cell with an immortalized cell; and
isolating the hybridoma.

15. The method according to claim 14, wherein the step of selecting a B cell further comprises the steps of:
incubating a B cell specific to the peptide within the MHC-peptide complex with a biotinylated MHC-peptide complex, wherein the MHC-peptide complex is capable of binding the B cell; and
binding the biotinylated MHC-peptide complex with an anti-biotin conjugated bead.

16. A hybridoma cell line having ATCC Accession No. PTA-10351.

17. A method of detecting the presence of at least one EBV infected cell in a subject, the method comprising:
contacting an antibody or fragment thereof according to claim 5 with a sample obtained from a subject; and
detecting binding of the antibody to the EBV infected cell.

18. A method of treating an EBV-linked disease, the method comprising administering to a subject in need thereof an antibody or a fragment thereof according to claim 5.

19. The method according to claim 18, wherein the EBV-linked disease is selected from the group consisting of lymphoproliferative disease, infectious mononucleosis, Nasopharyngeal Carcinoma, Burkitt's Lymphoma, and/or Hodgkins lymphoma.

20. A pharmaceutical composition comprising the antibody or a fragment thereof according to claim 5 for the treatment of an EBV-linked disease.

21. A kit for diagnosing an EBV-linked disease, the kit comprising the antibody or a fragment thereof according to claim 5.

22. An isolated nucleic acid molecule encoding:
at least one antibody heavy chain, wherein the heavy chain comprises an amino acid sequence selected from the group consisting of SEQ ID NOS:4 to 6, or a variant, mutant or fragment thereof; and/or
at least one antibody light chain, wherein the light chain comprises an amino acid sequence selected from the group consisting of SEQ ID NOS:1 to 3, or a variant, mutant or fragment thereof.

23. An expression vector comprising the nucleic acid of claim 22.

24. A host cell comprising the expression vector according to claim 23.


SEQUENCE LISTING (8 sequences)

SEQ ID NO: 1 - protein, 108 aa, Mus sp.
  Light chain variable region of antibody 1 specific to HLA-A201/LMP-1
DIVMTQSQKFMSTSVGDRVSITCKASQNVHTAVAWYQQKAGQSPKALIYLASNRHTGVPDRFTGSGSGTDFTLTISNVQSEDLADYFCLQHWNNPLTFGAGTKLELKR

SEQ ID NO: 2 - protein, 107 aa, Mus sp.
  Light chain variable region of antibody 2 specific to HLA-A201/LMP-1
DIVMTQSHKFMPTSVGDRVSITCKASQDVSTAVAWYQQKPGQSPKLLIHSASYRYTGVPDRFTGSGSGTDFTFTISSVQAEDLAVYYCQQHYTTRTFGGGTKLEIKR

SEQ ID NO: 3 - protein, 109 aa, Mus sp.
  Light chain variable region of antibody 3 specific to HLA-A201/LMP-1
DIFLTQSPAILSVSPGERVSFSCRASQSIGTSIHWYQQRTNGSPRLLIKYASESISGIPSRFSGSGSGTDFTLSINSVESEYIADYYCQQSNSWPTYTFGGGTKLEIKR

SEQ ID NO: 4 - protein, 117 aa, Mus sp.
  Heavy chain variable region of antibody 1 specific to HLA-A201/LMP-1
QVQLQQSGAELMKPGASVKISCKATGYTFSYYWIDWVKQRPGHGLEWIGEIFPGSNSTNYHENFRGKATLTADSSSNTAYMQLSSLASEDSAVYYCAREGTGYFDHWGQGTTLTVSS

SEQ ID NO: 5 - protein, 102 aa, Mus sp.
  Heavy chain variable region of antibody 2 specific to HLA-A201/LMP-1
DVQLVESGGGLVQPGGSRKLSCAASGFTFSSFGMHWVRQAPEKGLEWVAYISSGSSTLHYADTVKGRFTISRDNPKNTLFLQMKLPSLCYGLLGSRNLSHRL

SEQ ID NO: 6 - protein, 117 aa, Mus sp.
  Heavy chain variable region of antibody 3 specific to HLA-A201/LMP-1
QVQLKESGPGLVAPSQSLSITCTVSGFSLTGYGVNWVRQPPGKGLEWLGMIWGDGSTDYNSALKSRLSISKDNSKSQVFLKMNSLQTDDTARYYCARDPYGYIFDYWGQGTTLTVSS

SEQ ID NO: 7 - protein, 9 aa, Epstein-Barr virus
  LMP-1 peptide
YLLEMLWRL

SEQ ID NO: 8 - protein, 9 aa, Influenza A virus
  HLA-A201 restricted Influenza A peptide
GILGFVFTL